(19) United States
(12) Patent Application Publication
(10) Pub. No.: US 2022/0195064 A1
(43) Pub. Date: Jun. 23, 2022

(54) ANTI-CD371 ANTIBODIES AND USES THEREOF

(71) Applicants: MEMORIAL SLOAN-KETTERING CANCER CENTER, New York, NY (US); TRI-INSTITUTIONAL THERAPEUTICS DISCOVERY INSTITUTE, INC., New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Anthony Daniyan, Somers, NY (US); Renier J. Brentjens, Short Hills, NJ (US); Ivo C. Lorenz, Brooklyn, NY (US); Mary Ann Pohl, Astoria, NY (US)

(73) Assignees: MEMORIAL SLOAN-KETTERING CANCER CENTER, New York, NY (US); TRI-INSTITUTIONAL THERAPEUTICS DISCOVERY INSTITUTE, INC., New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 17/692,979

(22) Filed: Mar. 11, 2022

Related U.S. Application Data
(63) Continuation of application No. PCT/US2020/050380, filed on Sep. 11, 2020.
(60) Provisional application No. 62/936,913, filed on Nov. 18, 2019.
(60) Provisional application No. 62/900,118, filed on Sep. 13, 2019.

(51) Int. Cl. C07K 16/28; A61K 47/68; G01N 33/574

(52) U.S. Cl. CPC C07K 16/2896; A61K 47/6803; G01N 33/57492; C07K 2317/56; G01N 2333/70596; C07K 2317/92; C07K 2317/567; C07K 2317/31; C07K 2317/622; C07K 2317/565

(57) ABSTRACT

The presently disclosed subject matter provides antibodies or antigen-binding fragments thereof that bind to CD371 and methods of using such antibodies or antigen-binding fragments thereof same.

### CROSS-REFERENCE TO RELATED APPLICATIONS

This application is a continuation of International Application No. PCT/US2020/050380, filed Sep. 11, 2020, which claims priority to U.S. Provisional Application No. 62/900,118 filed Sep. 13, 2019 and U.S. Provisional Application No. 62/936,913 filed Nov. 18, 2019, the contents of each of which are incorporated by reference in their entireties herein, and to which each of which priority is claimed.

### SEQUENCE LISTING

This application contains a Sequence Listing which has been submitted in ASCII format via EFS-Web and is hereby incorporated by reference in its entirety. Said ASCII copy, created on Mar. 10, 2022, is named 0893390226_ST25.TXT and is 58,905 bytes in size.

### 1. FIELD OF THE INVENTION

The presently disclosed subject matter relates to antibodies that bind to CD371, and methods of using such antibodies.

### 2. BACKGROUND OF THE INVENTION

CD371 (CEC12A), also known as DCAL-2, MICL or CLL-1, is a 30 kD C-type lectin transmembrane glycoprotein. It is expressed on monocytes, granulocytes, natural killer (NK) cells, and basophils. CD371 is an immunoinhibitory receptor that recruits Src homology phosphatases SHP-1 and SHP-2 to its phosphorylated cytoplasmic immunoreceptor tyrosine-based inhibitory motif (ITIM) (Sancho et al., Annu Rev. Immunol (2012); 30:491-529; Yan et al., Front Immunol (2015); 6:408; Lahoud et al., J Immunol (2011); 187:842). CD371 has been implicated as a negative regulatory uric acid crystals (monosodium urate, MSU) receptor that controls autoimmunity and inflammatory disease (Neumann et al., Immunity (2014); 40:389-99). CD371 is a negative regulator of granulocyte and monocyte function (Marshall et al., J Biol Chem (2004); 279(15):14792-802; Pye et al., Eur J Immunol (2008); 38(4):1157-63). Abberant expression of CD371 has been reported in acute myeloid leukaemia (AML) and myelodysplastic syndrome (MDS) (Sadonik et al., Blood (2016); 128:4234; Toft-Petersen et al., Br J Haematol (2016); 175(3):393-41). Recent study shows that CD371 is expressed on 92% acute myeloid leukemia (AML) and absent on granulocyte-macrophage progenitors (GMPs) (Bakker et al., Cancer Res. (2004); 64(22):8443-50). CD371 is also expressed on leukemic stem cell (LSC), which possesses the ability to indefinitely self-renew and produce plenty of daughter blast cells with a specific phenotype of CD371, acting as one of most important reasons of leukemia relapse (Siveen et al., Mol Cancer (2017); 16:13; Yoshida et al., Cancer Sci (2016); 107:5-11). Given the significant role for CD371 in diseases, antibodies that bind to CD371 and methods of using such agents, are desired.

### 3. SUMMARY OF THE INVENTION

The presently disclosed subject matter provides antibodies or antigen-binding fragments thereof that specifically bind to CD371, and methods of using the antibodies or antigen-binding fragments thereof.

In certain embodiments, the anti-CD371 antibody or an antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 1, SEQ ID NO: 3, SEQ ID NO: 5, SEQ ID NO: 7, SEQ ID NO: 9, or SEQ ID NO: 11.

In certain embodiments, the anti-CD371 antibody or an antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 2, SEQ ID NO: 4, SEQ ID NO: 6, SEQ ID NO: 8, SEQ ID NO: 10, or SEQ ID NO: 12.

In certain embodiments, the anti-CD371 antibody or an antigen-binding fragment thereof comprises (a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 1, SEQ ID NO: 3, SEQ ID NO: 5, SEQ ID NO: 7, SEQ ID NO: 9, or SEQ ID NO: 11; and (b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 2, SEQ ID NO: 4, SEQ ID NO: 6, SEQ ID NO: 8, SEQ ID NO: 10, or SEQ ID NO: 12.

In certain embodiments, the heavy chain variable region and the light chain variable region of the anti-CD371 antibody or antigen-binding fragment thereof are selected from the group consisting of:

(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 1, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 2;

(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 3, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 4;

(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 5, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 6;

(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8;

(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10; and

(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 11, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 12.

In certain embodiments, the anti-CD371 antibody or antigen-binding fragment thereof comprises a heavy chain variable region comprising the amino acid sequence set forth in SEQ ID NO: 1, SEQ ID NO: 3, SEQ ID NO: 5, SEQ ID NO: 7, SEQ ID NO: 9, or SEQ ID NO: 11. In certain embodiments, the anti-CD371 antibody or antigen-binding fragment thereof, comprises a light chain variable region comprising the amino acid sequence set forth in SEQ ID NO: 2, SEQ ID NO: 4, SEQ ID NO: 6, SEQ ID NO: 8, SEQ ID NO: 10, or SEQ ID NO: 12.

In certain embodiments, the anti-CD371 antibody or antigen-binding fragment thereof comprises a heavy chain variable region comprising the amino acid sequence set forth in SEQ ID NO: 1, SEQ ID NO: 3, SEQ ID NO: 5, SEQ ID NO: 7, SEQ ID NO: 9, or SEQ ID NO: 11; and light chain variable region comprising the amino acid sequence set forth in SEQ ID NO: 2, SEQ ID NO: 4, SEQ ID NO: 6, SEQ ID NO: 8, SEQ ID NO: 10, or SEQ ID NO: 12.

In certain embodiments, the anti-CD371 antibody or antigen-binding fragment thereof comprises a heavy chain variable region and a light chain variable region, wherein the heavy chain variable region and the light chain variable region are selected from the group consisting of:

(a) a heavy chain variable region comprising the amino acid sequence set forth in SEQ ID NO: 1, and a light chain variable region comprising the amino acid sequence set forth in SEQ ID NO: 2;

(b) a heavy chain variable region comprising the amino acid sequence set forth in SEQ ID NO: 3, and a light chain variable region comprising the amino acid sequence set forth in SEQ ID NO: 4;

(c) a heavy chain variable region comprising the amino acid sequence set forth in SEQ ID NO: 5, and a light chain variable region comprising the amino acid sequence set forth in SEQ ID NO: 6;

(d) a heavy chain variable region comprising the amino acid sequence set forth in SEQ ID NO: 7, and a light chain variable region comprising the amino acid sequence set forth in SEQ ID NO: 8;

(e) a heavy chain variable region comprising the amino acid sequence set forth in SEQ ID NO:9, and a light chain variable region comprising the amino acid sequence set forth in SEQ ID NO:10; and

(f) a heavy chain variable region comprising the amino acid sequence set forth in SEQ ID NO: 11, and a light chain variable region comprising the amino acid sequence set forth in SEQ ID NO: 12.

In certain embodiments, the anti-CD371 antibody or antigen-binding fragment thereof comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:

(a) a heavy chain variable region CDR3 comprising the amino acid sequence set forth in SEQ ID NO: 30 and a conservative modification thereof, and a light chain variable region CDR3 comprising the amino acid sequence set forth in SEQ ID NO: 33 and a conservative modification thereof;

(b) a heavy chain variable region CDR3 comprising the amino acid sequence set forth in SEQ ID NO: 36 and a conservative modification thereof, and a light chain variable region CDR3 comprising the amino acid sequence set forth in SEQ ID NO: 39 and a conservative modification thereof;

(c) a heavy chain variable region CDR3 comprising the amino acid sequence set forth in SEQ ID NO: 42 and a conservative modification thereof, and a light chain variable region CDR3 comprising the amino acid sequence set forth in SEQ ID NO: 45 and a conservative modification thereof;

(d) a heavy chain variable region CDR3 comprising the amino acid sequence set forth in SEQ ID NO: 48 and a conservative modification thereof, and a light chain variable region CDR3 comprising the amino acid sequence set forth in SEQ ID NO: 51 and a conservative modification thereof;

(e) a heavy chain variable region CDR3 comprising the amino acid sequence set forth in SEQ ID NO: 54 and a conservative modification thereof, and a light chain variable region CDR3 comprising the amino acid sequence set forth in SEQ ID NO: 57 and a conservative modification thereof, and

(f) a heavy chain variable region CDR3 comprising the amino acid sequence set forth in SEQ ID NO: 60 and a conservative modification thereof, and a light chain variable region CDR3 comprising the amino acid sequence set forth in SEQ ID NO: 63 and a conservative modification thereof.

In certain embodiments, the heavy chain variable region and light chain variable region CDR2 domains of the antibody or antigen-binding portion thereof are selected from the group consisting of:

(a) a heavy chain variable region CDR2 comprising the amino acid sequence set forth in SEQ ID NO: 29 and a conservative modification thereof, and a light chain variable region CDR2 comprising the amino acid sequence set forth in SEQ ID NO: 32 and a conservative modification thereof;

(b) a heavy chain variable region CDR2 comprising the amino acid sequence set forth in SEQ ID NO: 35 and a conservative modification thereof, and a light chain variable region CDR2 comprising the amino acid sequence set forth in SEQ ID NO: 38 and a conservative modification thereof;

(c) a heavy chain variable region CDR2 comprising the amino acid sequence set forth in SEQ ID NO: 41 and a conservative modification thereof, and a light chain variable region CDR2 comprising the amino acid sequence set forth in SEQ ID NO: 44 and a conservative modification thereof;

(d) a heavy chain variable region CDR2 comprising the amino acid sequence set forth in SEQ ID NO: 47 and a conservative modification thereof, and a light chain variable region CDR2 comprising the amino acid sequence set forth in SEQ ID NO: 50 and a conservative modification thereof;

(e) a heavy chain variable region CDR2 comprising the amino acid sequence set forth in SEQ ID NO: 53 and a conservative modification thereof, and a light chain variable region CDR2 comprising the amino acid sequence set forth in SEQ ID NO: 56 and a conservative modification thereof, and

(f) a heavy chain variable region CDR2 comprising the amino acid sequence set forth in SEQ ID NO: 59 and a conservative modification thereof, and a light chain variable region CDR2 comprising the amino acid sequence set forth in SEQ ID NO: 62 and a conservative modification thereof.

In certain embodiments, the anti-CD371 heavy chain variable region and light chain variable region CDR1 domains of the antibody or antigen-binding portion thereof are selected from the group consisting of:

(a) a heavy chain variable region CDR1 comprising the amino acid sequence set forth in SEQ ID NO: 28 and a conservative modification thereof, and a light chain variable region CDR1 comprising the amino acid sequence set forth in SEQ ID NO: 31 and a conservative modification thereof;

(b) a heavy chain variable region CDR1 comprising the amino acid sequence set forth in SEQ ID NO: 34 and a conservative modification thereof, and a light chain variable region CDR1 comprising the amino acid sequence set forth in SEQ ID NO: 37 and a conservative modification thereof;

(c) a heavy chain variable region CDR1 comprising the amino acid sequence set forth in SEQ ID NO: 40 and a conservative modification thereof, and a light chain variable region CDR1 comprising the amino acid sequence set forth in SEQ ID NO: 43 and a conservative modification thereof;

(d) a heavy chain variable region CDR1 comprising the amino acid sequence set forth in SEQ ID NO: 46 and a conservative modification thereof, and a light chain variable region CDR1 comprising the amino acid sequence set forth in SEQ ID NO: 49 and a conservative modification thereof;

(e) a heavy chain variable region CDR1 comprising the amino acid sequence set forth in SEQ ID NO: 52 and a conservative modification thereof, and a light chain variable region CDR1 comprising the amino acid sequence set forth in SEQ ID NO: 55 and a conservative modification thereof, and

(f) a heavy chain variable region CDR1 comprising the amino acid sequence set forth in SEQ ID NO: 58 and a conservative modification thereof, and a light chain variable region CDR1 comprising the amino acid sequence set forth in SEQ ID NO: 61 and a conservative modification thereof.

In certain embodiments, one or more of the CDR sequences have up to about 5 amino acid substitutions. In certain embodiments, one or more of the CDR sequences have up to about 3 amino acid substitutions.

In certain embodiments, the anti-CD371 antibody or antigen-binding fragment thereof comprises:

(a) a heavy chain variable region CDR1 comprising the amino acid sequence set forth in SEQ ID NO: 28; a heavy chain variable region CDR2 comprising the amino acid sequence set forth in SEQ ID NO: 29; and a heavy chain variable region CDR3 comprising the amino acid sequence set forth in SEQ ID NO: 30;

(b) a heavy chain variable region CDR1 comprising the amino acid sequence set forth in SEQ ID NO: 34; a heavy chain variable region CDR2 comprising the amino acid sequence set forth in SEQ ID NO: 35; and a heavy chain variable region CDR3 comprising the amino acid sequence set forth in SEQ ID NO: 36;

(c) a heavy chain variable region CDR1 comprising the amino acid sequence set forth in SEQ ID NO: 40; a heavy chain variable region CDR2 comprising the amino acid sequence set forth in SEQ ID NO: 41; and a heavy chain variable region CDR3 comprising the amino acid sequence set forth in SEQ ID NO: 42;

(d) a heavy chain variable region CDR1 comprising the amino acid sequence set forth in SEQ ID NO: 46; a heavy chain variable region CDR2 comprising the amino acid sequence set forth in SEQ ID NO: 47; and a heavy chain variable region CDR3 comprising the amino acid sequence set forth in SEQ ID NO: 48;

(e) a heavy chain variable region CDR1 comprising the amino acid sequence set forth in SEQ ID NO: 52; a heavy chain variable region CDR2 comprising the amino acid sequence set forth in SEQ ID NO: 53; and a heavy chain variable region CDR3 comprising the amino acid sequence set forth in SEQ ID NO: 54, or

(f) a heavy chain variable region CDR1 comprising the amino acid sequence set forth in SEQ ID NO: 58; a heavy chain variable region CDR2 comprising the amino acid sequence set forth in SEQ ID NO: 59; and a heavy chain variable region CDR3 comprising the amino acid sequence set forth in SEQ ID NO: 60.

In certain embodiments, the anti-CD371 antibody or antigen-binding fragment thereof comprises:

(a) a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33;

(b) a light chain variable region CDR1 comprising the amino acid sequence set forth in SEQ ID NO: 37; a light chain variable region CDR2 comprising the amino acid sequence set forth in SEQ ID NO: 38; and a light chain variable region CDR3 comprising the amino acid sequence set forth in SEQ ID NO: 39;

(c) a light chain variable region CDR1 comprising the amino acid sequence set forth in SEQ ID NO: 43; a light chain variable region CDR2 comprising the amino acid sequence set forth in SEQ ID NO: 44; and a light chain variable region CDR3 comprising the amino acid sequence set forth in SEQ ID NO: 45;

(d) a light chain variable region CDR1 comprising the amino acid sequence set forth in SEQ ID NO: 49; a light chain variable region CDR2 comprising the amino acid sequence set forth in SEQ ID NO: 50; and a light chain variable region CDR3 comprising the amino acid sequence set forth in SEQ ID NO: 51;

(e) a light chain variable region CDR1 comprising the amino acid sequence set forth in SEQ ID NO: 55; a light chain variable region CDR2 comprising the amino acid sequence set forth in SEQ ID NO: 56; and a light chain variable region CDR3 comprising the amino acid sequence set forth in SEQ ID NO: 57, or

(f) a light chain variable region CDR1 comprising the amino acid sequence set forth in SEQ ID NO: 61; a light chain variable region CDR2 comprising the amino acid sequence set forth in SEQ ID NO: 62; and a light chain variable region CDR3 comprising the amino acid sequence set forth in SEQ ID NO: 63.

In certain embodiments, the anti-CD371 antibody or antigen-binding fragment thereof comprises:

(a) a heavy chain variable region CDR1 comprising the amino acid sequence set forth in SEQ ID NO: 28; a heavy chain variable region CDR2 comprising the amino acid sequence set forth in SEQ ID NO: 29; a heavy chain variable region CDR3 comprising the amino acid sequence set forth in SEQ ID NO: 30; a light chain variable region CDR1 comprising the amino acid sequence set forth in SEQ ID NO: 31; a light chain variable region CDR2 comprising the amino acid sequence set forth in SEQ ID NO: 32; and a light chain variable region CDR3 comprising the amino acid sequence set forth in SEQ ID NO: 33;

(b) a heavy chain variable region CDR1 comprising the amino acid sequence set forth in SEQ ID NO: 34; a heavy chain variable region CDR2 comprising an amino acid sequence set forth in SEQ ID NO: 35; a heavy chain variable region CDR3 comprising the amino acid sequence set forth in SEQ ID NO: 36; a light chain variable region CDR1 comprising the amino acid sequence set forth in SEQ ID NO: 37; a light chain variable region CDR2 comprising the amino acid sequence set forth in SEQ ID NO: 38; and a light chain variable region CDR3 comprising the amino acid sequence set forth in SEQ ID NO: 39;

(c) a heavy chain variable region CDR1 comprising the amino acid sequence set forth in SEQ ID NO: 40; a heavy chain variable region CDR2 comprising the amino acid sequence set forth in SEQ ID NO: 41; a heavy chain variable region CDR3 comprising the amino acid sequence set forth in SEQ ID NO: 42; a light chain variable region CDR1 comprising the amino acid sequence set forth in SEQ ID NO: 43; a light chain variable region CDR2 comprising the amino acid sequence set forth in SEQ ID NO: 44; and a light chain variable region CDR3 comprising the amino acid sequence set forth in SEQ ID NO: 45;

(d) a heavy chain variable region CDR1 comprising the amino acid sequence set forth in SEQ ID NO: 46; a heavy chain variable region CDR2 comprising the amino acid sequence set forth in SEQ ID NO: 47; a heavy chain variable region CDR3 comprising the amino acid sequence set forth in SEQ ID NO: 48; a light chain variable region CDR1 comprising the amino acid sequence set forth in SEQ ID NO: 49; a light chain variable region CDR2 comprising the amino acid sequence set forth in SEQ ID NO: 50; and a light chain variable region CDR3 comprising the amino acid sequence set forth in SEQ ID NO: 51;

(e) a heavy chain variable region CDR1 comprising the amino acid sequence set forth in SEQ ID NO: 52; a heavy chain variable region CDR2 comprising the amino acid sequence set forth in SEQ ID NO: 53; a heavy chain variable region CDR3 comprising the amino acid sequence set forth in SEQ ID NO: 54; a light chain variable region CDR1 comprising the amino acid sequence set forth in SEQ ID NO: 55; a light chain variable region CDR2 comprising the amino acid sequence set forth in SEQ ID NO: 56; and a light chain variable region CDR3 comprising the amino acid sequence set forth in SEQ ID NO: 57; or

(f) heavy chain variable region CDR1 comprising the amino acid sequence set forth in SEQ ID NO: 58; a heavy chain variable region CDR2 comprising the amino acid sequence set forth in SEQ ID NO: 59; a heavy chain variable region CDR3 comprising the amino acid sequence set forth in SEQ ID NO: 60; a light chain variable region CDR1 comprising the amino acid sequence set forth in SEQ ID NO: 61; a light chain variable region CDR2 comprising the amino acid sequence set forth in SEQ ID NO: 62; and a light chain variable region CDR3 comprising the amino acid sequence set forth in SEQ ID NO: 63.

The presently disclosed subject matter provides an antibody or an antigen-binding fragment thereof, which cross-competes for binding to CD371 with any of the above-described antibody or an antigen-binding fragment thereof.

The presently disclosed subject matter provides an antibody or an antigen-binding fragment thereof, which binds to the same epitope on CD371 with any of the above-described antibody or an antigen-binding fragment thereof.

In certain embodiments, the sequence of the antibody is in a light-heavy variable chain orientation (V_{L}-V_{H}). In certain embodiments, the antibody or antigen-binding fragment thereof binds to human CD371 with a dissociation constant (K_{D}) of between about 1×10⁻⁷ M and about 1×10⁻⁸ M or between about 1×10⁻⁹ M and about 1×10⁻⁸ M.

In certain embodiments, the antibody or antigen-binding fragment thereof comprises the amino acid sequence set forth in SEQ ID NO: 16, SEQ ID NO: 17, SEQ ID NO: 18, SEQ ID NO: 19, SEQ ID NO: 20, or SEQ ID NO: 21.

In certain embodiments, the antibody or antigen-binding fragment thereof comprises a human variable region framework region. In certain embodiments, the antibody or antigen-binding fragment thereof is a fully human or an antigen-binding fragment thereof. In certain embodiments, the antibody or antigen-binding fragment thereof is a chimeric antibody or an antigen-binding fragment thereof. In certain embodiments, the antibody or antigen-binding portion thereof is a humanized antibody or an antigen-binding fragment thereof. In certain embodiments, the antigen-binding fragment of the antibody is a Fab, Fab′, F(ab′)₂, variable fragment (Fv) or a single chain variable fragment (scFv).

The presently disclosed subject matter also provides an immunoconjugate comprising the antibody or antigen-binding fragment thereof disclosed herein, linked to a therapeutic agent. In certain embodiments, the therapeutic agent is a drug, a cytotoxin, or a radioactive isotope.

Furthermore, the presently disclosed subject matter provides a bispecific molecule comprising the antibody or antigen-binding fragment thereof disclosed herein, linked to a second functional moiety. In certain embodiments, the second functional moiety has a different binding specificity than the antibody or antigen binding fragment thereof.

The presently disclosed subject matter also provides a composition comprising the antibody or antigen-binding fragment thereof disclosed herein, the immunoconjugate disclosed herein, or the bispecific antibody disclosed herein. In certain embodiments, the composition is a pharmaceutical composition that further comprises a pharmaceutically acceptable carrier.

In addition, the presently disclosed subject matter provides a nucleic acid that encodes the antibody or antigen-binding fragment thereof disclosed herein, an expression vector comprising such nucleic acid molecule, and a host cell comprising such expression vector.

The presently disclosed subject matter provides a method for detecting CD371 in a whole cell or tissue. In certain embodiments, the method comprises: contacting a cell or tissue with the antibody or antigen-binding fragment thereof disclosed herein, wherein said antibody or antigen-binding fragment thereof comprises a detectable label; and determining the amount of the labeled antibody or antigen-binding fragment thereof bound to said cell or tissue by measuring the amount of detectable label associated with said cell or tissue, wherein the amount of bound antibody or antigen-binding fragment thereof indicates the amount of CD371 in said cell or tissue.

Furthermore, the presently disclosed subject matter provides methods of treating a tumor burden in a subject. In certain embodiments, the method comprises administering to the subject an antibody or antigen-binding fragment thereof, the immunoconjugate thereof, the bispecific molecule thereof, or the composition disclosed herein. In certain embodiments, the method reduces the number of the tumor cells. In certain embodiments, the method reduces the tumor size. In certain embodiments, the method eradicates the tumor in the subject. In certain embodiments, the subject is a human.

Furthermore, the presently disclosed subject matter provides methods of treating and/or preventing a tumor or neoplasm in a subject. In certain embodiments, the method comprises administering to the subject an antibody or antigen-binding fragment thereof, the immunoconjugate thereof, the bispecific molecule thereof, or the composition thereof disclosed herein. In certain embodiments, the method eradicates the tumor in the subject. In certain embodiments, the subject is a human.

In addition, the presently disclosed subject matter provides a method of increasing or lengthening survival of a subject having a tumor or neoplasm. In certain embodiments, the method comprises administering to the subject an antibody or antigen-binding fragment thereof, the immunoconjugate thereof, the bispecific molecule thereof, or the composition thereof disclosed herein. In certain embodiments, the method eradicates the tumor in the subject. In certain embodiments, the subject is a human.

In addition, the presently disclosed subject matter provides a method of preventing and/or treating a tumor or neoplasm. In certain embodiments, the method comprises administering to the subject an antibody or antigen-binding fragment thereof, the immunoconjugate thereof, the bispecific molecule thereof, or the composition thereof disclosed herein. In certain embodiments, the subject is a human.

In certain embodiments, the tumor or neoplasm is selected from the group consisting of acute myeloid leukemia (AML), multiple myeloma, Non-Hodgkin's Lymphoma, Hodgkin's Lymphoma, Chronic Lymphocytic Leukemia (CLL), glioblastoma, myelodysplastic syndrome (MDS), and chronic myelogenous leukemia (CML). In certain embodiments, the tumor is AML.

Furthermore, the presently disclosed subject matter provides a kit for treating a tumor burden in a subject, treating and/or preventing a tumor or neoplasm, and/or increasing or lengthening survival of a subject having a tumor or neoplasm, comprising the antibody or antigen-binding fragment thereof, the immunoconjugate thereof, the bispecific molecule thereof, or the composition thereof disclosed herein. In certain embodiments, the kit further comprises written instructions for using the antibody or antigen-binding fragment thereof, the immunoconjugate thereof, the bispecific molecule thereof, or the composition thereof disclosed herein for treating a tumor burden in a subject, treating and/or preventing a tumor or neoplasm, and/or increasing or lengthening survival of a subject having a tumor or neoplasm.

### 4. BRIEF DESCRIPTION OF THE FIGURES

The following Detailed Description, given by way of example, but not intended to limit the invention to specific embodiments described, may be understood in conjunction with the accompanying drawings.

FIG. 1 depicts binding of anti-CD371 monoclonal phage preps to HEK293H cells transfected with human CD371.

FIGS. 2A and 2B depict binding of 1B10 and 1C3 formatted as human IgG1 to OCI cells. FIG. 2A shows B10 (referred to as “1B10”). FIG. 2B shows C3 (or referred to as “1C3”).

FIG. 3 depicts binding of scFv-Fc fusion proteins and scFv fragments to HEK293 cells expressing human CD371.

### 5. DETAILED DESCRIPTION OF EMBODIMENTS OF THE INVENTION

The presently disclosed subject matter provides anti-CD371 antibodies. Non-limiting embodiments of the present disclosure are described by the present specification and Examples.

For purposes of clarity of disclosure and not by way of limitation, the detailed description is divided into the following subsections:
5.1. Definitions;
5.2. CD371;
5.3. Anti-CD371 Antibodies;
5.4. Nucleic Acids encoding the Antibodies or Antigen-binding Fragments;
5.5. Pharmaceutical Compositions and Methods of Treatment;
5.6. Kits; and
5.7. Methods of Detection.

### 5.1. Definitions

In the description that follows, certain conventions will be followed as regards the usage of terminology. Generally, terms used herein are intended to be interpreted consistently with the meaning of those terms as they are known to those of skill in the art.

An “antigen-binding protein” is a protein or polypeptide that comprises an antigen-binding region or antigen-binding portion, that is, has a strong affinity to another molecule to which it binds. Antigen-binding proteins encompass antibodies, chimeric antigen receptors (CARs) and fusion proteins.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding portion” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to CD371” is intended to refer to an antibody that binds to CD371 (e.g., human CD371) with a dissociation constant (K_{d}) of about 5×10⁻⁷ M or less, about 1×10⁻⁷ M or less, about 5×10⁻⁸ M or less, about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less.

An “antibody that competes for binding” or “antibody that cross-competes for binding” with a reference antibody for binding to an antigen, e.g., CD371, refers to an antibody that blocks binding of the reference antibody to the antigen (e.g., CD371) in a competition assay by 50% or more, and conversely, the reference antibody blocks binding of the antibody to the antigen (e.g., CD371) in a competition assay by 50% or more. An exemplary competition assay is described in “Antibodies”, Harlow and Lane (Cold Spring Harbor Press, Cold Spring Harbor, N.Y.).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen (e.g., a CD371 polypeptide).”

The term “antigen-binding portion” or “antigen-binding region” of an antibody, as used herein, refers to that region or portion of the antibody that binds to the antigen and which confers antigen specificity to the antibody; fragments of antigen-binding proteins, for example, antibodies includes one or more fragments of an antibody that retain the ability to specifically bind to an antigen (e.g., a CD391 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

An “antibody” or “antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises the amino acid sequence set forth in SEQ ID NO: 13, which is provided below:

| [SEQ ID NO: 13] | |
|---|---|
| | GGGGSGGGGSGGGSGGGGS |

In certain embodiments, the linker comprises the amino acid sequence set forth in SEQ ID NO: 14, which is provided below:

| [SEQ ID NO: 14] | |
|---|---|
| | GGGGSGGGGSGGGGS |

In certain embodiments, the linker comprises the amino acid sequence set forth in SEQ ID NO: 64, which is provided below:

| [SEQ ID NO: 64] | |
|---|---|
| | GGGGSGGGGSGGGGSGGGSGGGGS |

In certain embodiments, the linker comprises the amino acid sequence set forth in SEQ ID NO: 65, which is provided below:

| [SEQ ID NO: 65] | |
|---|---|
| | GGGGSGGGGSGGGGSGGGGSGGGSGGGGS |

In certain embodiments, the linker comprises the amino acid sequence set forth in SEQ ID NO: 66, which is provided below:

| [SEQ ID NO: 66] | |
|---|---|
| | GGGGS |

In certain embodiments, the linker comprises the amino acid sequence set forth in SEQ ID NO: 67, which is provided below:

| [SEQ ID NO: 67] | |
|---|---|
| | GGGGSGGGGS |

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 Aug. 12; Shieh et al., J Imunol 2009; 183(4):2277-85; Giomarelli et al., Thromb Haemost 2007; 97(6):955-63; Fife et al., J Clin Invst 2006; 116(8):2252-61; Brocks et al., Immunotechnology 1997; 3(3):173-84; Moosmayer et al., Ther Immunol 1995; 2(10:31-40). Agonistic scFvs having stimulatory activity have been described (see, e.g., Peter et al., JBioi Chem 2003; 25278(38):36740-7; Xie et al., Nat Biotech 1997; 15(8):768-71; Ledbetter et al., Crit Rev Immunol 1997; 17(5-6):427-55; Ho et al., BioChim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope. In certain embodiments, the CDRs are identified according to the IMGT system. In certain embodiments, the CDRs are identified using the IMGT numbering system accessible at http://www.imgt.org/IMGT_vquest/input.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-KLB antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “immunoconjugate” is an antibody conjugated to one or more heterologous molecule(s), including, but not limited to, a cytotoxic agent.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor (acute myeloid leukemia (AML)).

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells.

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 5.2. CD371

CD371 (CEC12A), also known as DCAL-2, MICL or CLL-1, is a 30 kD C-type lectin transmembrane glycoprotein. It is expressed on monocytes, granulocytes, natural killer (NK) cells, and basophils. CD371 is an immunoinhibitory receptor that recruits Src homology phosphatases SHP-1 and SHP-2 to its phosphorylated cytoplasmic immunoreceptor tyrosine-based inhibitory motif (ITIM) (Sancho et al., Annu Rev. Immunol (2012); 30:491-529; Yan et al., Front Immunol (2015); 6:408; Lahoud et al., J Immunol (2011); 187:842). CD371 has been implicated as a negative regulatory uric acid crystals (monosodium urate, MSU) receptor that controls autoimmunity and inflammatory disease (Neumann et al., Immunity (2014); 40:389-99). CD371 is a negative regulator of granulocyte and monocyte function (Marshall et al., J Biol Chem (2004); 279(15):14792-802; Pye et al., Eur J Immunol (2008); 38(4):1157-63).

In certain embodiments, CD371 is human CD371 comprising or consisting of the amino acid sequence with a NCBI Reference No: NP_612210.4 (SEQ ID NO: 15), or a fragment thereof.

SEQ ID NO: 15 is provided below:

[SEQ ID NO: 15]

MSEEVTYADL QFQNSSEMEK IPEIGKFGEK APPAPSHVWR
PAALFLTLLC LLLLIGLGVL ASMFHVTLKI EMKKMNKLQN
ISEELQRNIS LQLMSNMNIS NKIRNLSTTL QTIATKLCRE
LYSKEQEHKC KPCPRRWIWH KDSCYFLSDD VQTWQESKMA
CAAQNASLLK INNKNALEFI KSQSRSYDYW LGLSPEEDST
RGMRVDNIIN SSAWVIRNAP DLNNMYCGYI NRLYVQYYHC
TYKKRMICEK MANPVQLGST YFREA

In certain embodiments, the CD371 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 15 or a fragment thereof.

### 5.3. Anti-CD371 Antibodies

The antibodies of the presently disclosed subject matter are characterized by particular functional features or properties of the antibodies. For example, the antibodies bind specifically to CD371 (e.g., bind to human CD371).

In certain embodiments, a presently disclosed antibody or antigen-binding fragment binds to CD371 (e.g., human CD371) with a binding affinity, for example with a dissociation constant (K_{d}) of 1×10⁻⁶ M or less, e.g., about 1×10⁻⁷ M or less, about 1×10⁻⁸ M or less, about 1×10⁻⁹ M or less, about 1×10⁻¹⁰ M or less, or about 1×10⁻¹¹ M or less. In certain embodiments, a presently disclosed antibody or antigen-binding fragment binds to CD371 (e.g., human CD371) with a K_{d} of between about 1×10⁻⁸ M and about 1×10-7 M. In certain embodiments, a presently disclosed antibody or antigen-binding fragment binds to CD371 (e.g., human CD371) with a K_{d} of between about 1×10⁻⁹ M and about 1×10⁻⁸ M. In certain embodiments, a presently disclosed antibody or antigen-binding fragment binds to CD371 (e.g., human CD371) with a K_{d} of about 1.5×10⁻⁸ M or less. In certain embodiments, a presently disclosed antibody or antigen-binding fragment binds to CD371 (e.g., human CD371) with a K_{d} of about 1×10⁻⁸ M or less. In certain embodiments, a presently disclosed antibody or antigen-binding fragment binds to CD371 (e.g., human CD371) with a K_{d} of about 1×10⁻⁸ M. In certain embodiments, a presently disclosed antibody or antigen-binding fragment binds to CD371 (e.g., human CD371) with a K_{d} of about 9×10⁻⁹ M.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding portion (a Fab, F(ab′)2, Fv or a single chain Fv fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). In certain embodiments, the antibody light chain constant region is chosen from, e.g., kappa or lambda, particularly kappa.

### 5.3.1. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity and utility of the presently disclosed antibodies or antigen-binding fragments in targeting a CD371 polypeptide (e.g., human CD371).

The presently disclosed molecules are based on the identification and selection of single chain variable fragments (scFvs) using phage display, the amino acid sequence of which confers the molecules' specificity for a CD371 polypeptide of interest and forms the basis of all antigen binding proteins of the disclosure. The scFv, therefore, can be used to design a diverse array of “antibody” molecules, including, for example, full length antibodies, fragments thereof, such as Fab and F(ab′)₂, minibodies, fusion proteins, including scFv-Fc fusions, multivalent antibodies, that is, antibodies that have more than one specificity for the same antigen or different antigens, for example, bispecific antibodies, tribodies, etc. (see Cuesta et al., Multivalent antibodies: when design surpasses evolution. Trends in Biotechnology 28:355-362 2010).

In certain embodiments, the antigen-binding protein is a full length antibody, the heavy and light chains of an antibody of the presently disclosed subject matter can be full-length (e.g., an antibody can include at least one, or two, complete heavy chains, and at least one, and preferably two, complete light chains) or can include an antigen-binding fragment (a Fab, F(ab′)₂, Fv or scFv). In certain embodiments, the antibody heavy chain constant region is chosen from IgG1, IgG2, IgG3, IgG4, IgM, IgA1, IgA2, IgD, and IgE, etc. In certain embodiments, the immunoglobulin isotype is selected from IgG1, IgG2, IgG3, and IgG4. In certain embodiments, the immunoglobulin isotype is IgG1 (e.g., human IgG1). The choice of antibody isotype can depend on the immune effector function that the antibody is designed to elicit.

In constructing a recombinant immunoglobulin, appropriate amino acid sequences for constant regions of various immunoglobulin isotypes and methods for the production of a wide array of antibodies are known to those of skill in the art.

In certain embodiments, the anti-CD371 scFv is a scFv-Fc fusion protein or a full-length human IgG with V_{H} and V_{L} regions or CDRs selected from Table 1. In certain embodiments, the anti-CD371 scFv comprises a V_{H} comprising the amino acid sequence set forth in SEQ ID NO: 1. In certain embodiments, the anti-CD371 scFv comprises a V_{L} comprising the amino acid sequence set forth in SEQ ID NO: 2. In certain embodiments, the scFv is designed as “B031_P1_PH1B10” (also referred to as “B10”).

In certain embodiments, the anti-CD371 scFv comprises a V_{H} comprising the amino acid sequence set forth in SEQ ID NO: 1 and a V_{L} comprising the amino acid sequence set forth in SEQ ID NO: 2. In certain embodiments, the anti-CD371 scFv comprises a V_{H} CDR1 comprising the amino acid sequence set forth in SEQ ID NO: 28 or a conservative modification thereof, a V_{H} CDR2 comprising the amino acid sequence set forth in SEQ ID NO: 29 or a conservative modification thereof, and a V_{H} CDR3 comprising the amino acid sequence set forth in SEQ ID NO: 30 or a conservative modification thereof. SEQ ID NOs: 28-30 are provided in Table 1.

In certain embodiments, the anti-CD371 scFv comprises a V_{L} CDR1 comprising the amino acid sequence set forth in SEQ ID NO: 31 or a conservative modification thereof, a V_{L} CDR2 comprising the amino acid sequence set forth in SEQ ID NO: 32 or a conservative modification thereof, and a V_{L} CDR3 comprising the amino acid sequence set forth in SEQ ID NO: 33 or a conservative modification thereof. SEQ ID NOs: 31-33 are provided in Table 1.

In certain embodiments, the anti-CD371 scFv comprises a V_{H} CDR1 comprising the amino acid sequence set forth in SEQ ID NO: 28 or a conservative modification thereof, a V_{H} CDR2 comprising the amino acid sequence set forth in SEQ ID NO: 29 or a conservative modification thereof, a V_{H} CDR3 comprising the amino acid sequence set forth in SEQ ID NO: 30 or a conservative modification thereof, a V_{L} CDR1 comprising the amino acid sequence set forth in SEQ ID NO: 31 or a conservative modification thereof, a V_{L} CDR2 comprising the amino acid sequence set forth in SEQ ID NO: 32 or a conservative modification, and a V_{L} CDR3 comprising the amino acid sequence set forth in SEQ ID NO: 33 or a conservative modification thereof.

In certain embodiments, the anti-CD371 scFv comprises a V_{H} CDR1 comprising the amino acid sequence set forth in SEQ ID NO: 28, a V_{H} CDR2 comprising the amino acid sequence set forth in SEQ ID NO: 29, a V_{H} CDR3 comprising the amino acid sequence set forth in SEQ ID NO: 30, a V_{L} CDR1 comprising the amino acid sequence set forth in SEQ ID NO: 31, a V_{L} CDR2 comprising the amino acid sequence set forth in SEQ ID NO: 32, and a V_{L} CDR3 comprising the amino acid sequence set forth in SEQ ID NO: 33.

In certain embodiments, the anti-CD371 scFv comprises a V_{H} comprising the amino acid sequence set forth in SEQ ID NO: 1, and a V_{L} comprising the amino acid sequence set forth in SEQ ID NO: 2. In certain embodiments, the V_{H} and V_{L} are linked via a linker. In certain embodiments, the linker comprises the amino acid sequence set forth in SEQ ID NO: 13.

In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, the anti-CD371 scFv comprises the amino acid sequence set forth in SEQ ID NO: 68, which is provided in Table 1.

In certain embodiments, a light chain variable region (V_{L}) is positioned at the N-terminus. In certain embodiments, the variable regions are positioned from the N- to the C-terminus: V_{L}-V_{H}. In certain embodiments, anti-CD371 scFv comprises the amino acid sequence set forth in SEQ ID NO: 16. An exemplary nucleotide sequence encoding the amino acid sequence of SEQ ID NO: 16 is set forth in SEQ ID NO: 22. SEQ ID NOS: 16 and 22 are provided in Table 1 below.

TABLE 1

| | CDRs | | |
|---|---|---|---|
| | 1 | 2 | 3 |
| V_{H} | GFTFSDYQ [SEQ ID | IQGGGGST [SEQ ID | AREMWRGDYYSGMDV [SEQ |
| | NO: 28] | NO: 29] | ID NO: 30] |
| V_{L} | QSVLDSYNNENN [SEQ | WAS [SEQ ID NO: | QQYTSEPIT [SEQ ID |
| | ID NO: 31] | 32] | NO: 33] |

| | |
|---|---|
| Full | EVQLLESGGGLVQPGGSLRLSCAASGFTFSDYQMSWVRQAPGKGLEWVSGIQGGGGSTYYA |
| V_{H} | DSVKGRFTISRDNSKNTLYLQMNSLRAEDTAVYYCAREMWRGDYYSGMDVWGQGTTVTVSS |
| | [SEQ ID NO: 1] |
| Full | DIVMTQSPDSLAVSLGERATINCKSSQSVLDSYNNENNLAWYQQKPGQPPKLLIYWASTRE |
| V_{L} | SGVPDRFSGSGSGTDFTLTISSLQAEDVAVYYCQQYTSEPITFGQGTKVEIK [SEQ ID |
| | NO: 2] |
| V_{L}-V_{H} | DIVMTQSPDSLAVSLGERATINCKSSQSVLDSYNNENNLAWYQQKPGQPPKLLIYWASTRE |
| scFv | SGVPDRFSGSGSGTDFTLTISSLQAEDVAVYYCQQYTSEPITFGQGTKVEIKGGGGSGGGG |
| | SGGGSGGGGSEVQLLESGGGLVQPGGSLRLSCAASGFTFSDYQMSWVRQAPGKGLEWVSGI |
| | QGGGGSTYYADSVKGRFTISRDNSKNTLYLQMNSLRAEDTAVYYCAREMWRGDYYSGMDVW |
| | GQGTTVTVSS [SEQ ID NO: 16] |
| DNA | GACATCGTGATGACCCAGTCTCCAGACTCCCTGGCTGTGTCTCTGGGCGAGCGTGCCACCA |
| for | TCAACTGCAAGTCCAGCCAGAGTGTTTTAGACAGCTATAACAATGAGAACAATTTAGCTTG |
| V_{L}-V_{H} | GTATCAGCAGAAACCAGGACAGCCTCCTAAGCTGCTCATTTACTGGGCATCTACCCGGGAA |
| scFv | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATACCAGCGAACCTAT |
| | CACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTAGCGA |
| | CTATCAGATGAGCTGGGTCCGCCAGGCTCCAGGGAAGGGGCTGGAGTGGGTGTCAGGCATT |
| | CAGGGTGGCGGTGGTAGCACATATTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGAGAGAGATGTGGCGTGGGGACTACTACTCCGGTATGGACGTCTGG |
| | GGCCAGGGGACCACGGTCACCGTCTCCTCA [SEQ ID NO: 22] |
| V_{H}-V_{L} | EVQLLESGGGLVQPGGSLRLSCAASGFTFSDYQMSWVRQAPGKGLEWVSGIQGGGGSTYYA |
| scFv | DSVKGRFTISRDNSKNTLYLQMNSLRAEDTAVYYCAREMWRGDYYSGMDVWGQGTTVTVSS |
| | GGGGSGGGGSGGGSGGGGSDIVMTQSPDSLAVSLGERATINCKSSQSVLDSYNNENNLAWY |
| | QQKPGQPPKLLIYWASTRESGVPDRFSGSGSGTDFTLTISSLQAEDVAVYYCQQYTSEPIT |
| | FGQGTKVEIK [SEQ ID NO: 68] |

In certain embodiments, the anti-CD371 scFv comprises a V_{H} comprising the amino acid sequence set forth in SEQ ID NO: 3 and a V_{L} comprising the amino acid sequence set forth in SEQ ID NO: 4, optionally with a linker sequence, for example a linker peptide, between the heavy chain variable region and the light chain variable region. In certain embodiments, the linker comprises the amino acid sequence set forth in SEQ ID NO: 13. SEQ ID NOs: 3 and 4 are provided in Table 2 below. In certain embodiments, the scFv is designated as “B031_P1_PH1C3” (also referred to as “C3”).

In certain embodiments, the anti-CD371 scFv is a scFv-Fc fusion protein or a full-length human IgG with V_{H} and V_{L} regions or CDRs selected from Table 2. In certain embodiments, the anti-CD371 scFv comprises a V_{H} comprising the amino acid sequence set forth in SEQ ID NO: 3, as shown in Table 2. In certain embodiments, the anti-CD371 scFv comprises a V_{L} comprising the amino acid sequence set forth in SEQ ID NO: 4. In certain embodiments, the anti-CD371 scFv comprises a V_{H} comprising the amino acid sequence set forth in SEQ ID NO: 3 and a V_{L} comprising the amino acid sequence set forth in SEQ ID NO: 4.

In certain embodiments, the anti-CD371 scFv comprises a V_{H} CDR1 comprising the amino acid sequence set forth in SEQ ID NO: 34 or a conservative modification thereof, a V_{H} CDR2 comprising the amino acid sequence set forth in SEQ ID NO: 35 or a conservative modification thereof, and a V_{H} CDR3 comprising the amino acid sequence set forth in SEQ ID NO: 36 or a conservative modification thereof. SEQ ID NOs: 34-36 are provided in Table 2.

In certain embodiments, the anti-CD371 scFv comprises a V_{L} CDR1 comprising the amino acid sequence set forth in SEQ ID NO: 37 or a conservative modification thereof, a V_{L} CDR2 comprising the amino acid sequence set forth in SEQ ID NO: 38 or a conservative modification thereof, and a V_{L} CDR3 comprising the amino acid sequence set forth in SEQ ID NO: 39 or a conservative modification thereof. SEQ ID NOs: 37-39 are provided in Table 2.

In certain embodiments, the anti-CD371 scFv comprises a V_{H} CDR1 comprising the amino acid sequence set forth in SEQ ID NO: 34 or a conservative modification thereof, a V_{H} CDR2 comprising the amino acid sequence set forth in SEQ ID NO: 35 or a conservative modification thereof, a V_{H} CDR3 comprising the amino acid sequence set forth in SEQ ID NO: 36 or a conservative modification thereof, a V_{L} CDR1 comprising the amino acid sequence set forth in SEQ ID NO: 37 or a conservative modification thereof, a V_{L} CDR2 comprising the amino acid sequence set forth in SEQ ID NO: 38 or a conservative modification thereof, and a V_{L} CDR3 comprising the amino acid sequence set forth in SEQ ID NO: 39 or a conservative modification.

In certain embodiments, the anti-CD371 scFv comprises a V_{H} CDR1 comprising the amino acid sequence set forth in SEQ ID NO: 34, a V_{H} CDR2 comprising the amino acid sequence set forth in SEQ ID NO: 35, a V_{H} CDR3 comprising the amino acid sequence set forth in SEQ ID NO: 36, a V_{L} CDR1 comprising the amino acid sequence set forth in SEQ ID NO: 37, a V_{L} CDR2 comprising the amino acid sequence set forth in SEQ ID NO: 38, and a V_{L} CDR3 comprising the amino acid sequence set forth in SEQ ID NO: 39.

In certain embodiments, the anti-CD371 scFv comprises a V_{H} comprising the amino acid sequence set forth in SEQ ID NO: 3, and a V_{L} comprising the amino acid sequence set forth in SEQ ID NO: 4. In certain embodiments, the V_{H} and V_{L} are linked via a linker. In certain embodiments, the linker comprises the amino acid sequence set forth in SEQ ID NO: 13.

In certain embodiments, a heavy chain variable region (V_{H}) is positioned at the N-terminus. In certain embodiments, the variable regions are positioned from the N- to the C-terminus: V_{H}-V_{L}. In certain embodiments, the anti-CD371 scFv comprises the amino acid sequence set forth in SEQ ID NO: 69, which is provided in Table 2.

In certain embodiments, a light chain variable region (V_{L}) is positioned at the N-terminus. In certain embodiments, the variable regions are positioned from the N- to the C-terminus: V_{L}-V_{H}. In certain embodiments, the anti-CD371 scFv comprises the amino acid sequence set forth in SEQ ID NO: 17. An exemplary nucleotide sequence encoding the amino acid sequence of SEQ ID NO: 17 is set forth in SEQ ID NO: 23. SEQ ID NOS: 17 and 23 are provided in Table 2 below.

TABLE 2

| | CDRs | | |
|---|---|---|---|
| | 1 | 2 | 3 |
| V_{H} | GFTFTSYA [SEQ ID | IDGSGGGT [SEQ ID | ARAYYDIL [SEQ ID NO: |
| | NO: 34] | NO: 35] | 36] |
| V_{L} | QSVLSSYNNENN | AAS [SEQ ID NO: | QQYYSEPYT [SEQ ID |
| | [SEQ ID NO: 37] | 38] | NO: 39] |

| | |
|---|---|
| Full | EVQLLESGGGLVQPGGSLRLSCAASGFTFTSYAMSWVRQAPGKGLEWVSGIDGSGGGTNYA |
| V_{H} | DSVKGRFTISRDNSKNTLYLQMNSLRAEDTAVYYCARAYYDILTGYPVDGMDVWGQGTTVT |
| | VSS [SEQ ID NO: 3] |
| Full | DIVMTQSPDSLAVSLGERATINCKSSQSVLSSYNNENNLAWYQQKPGQPPKLLIYAASTRE |
| V_{L} | SGVPDRFSGSGSGTDFTLTISSLQAEDVAVYYCQQYYSEPYTFGQGTKVEIK [SEQ ID |
| | NO: 4] |
| V_{L}-V_{H} | DIVMTQSPDSLAVSLGERATINCKSSQSVLSSYNNENNLAWYQQKPGQPPKLLIYAASTRE |
| scFv | SGVPDRFSGSGSGTDFTLTISSLQAEDVAVYYCQQYYSEPYTFGQGTKVEIKGGGGSGGGG |
| | SGGGSGGGGSEVQLLESGGGLVQPGGSLRLSCAASGFTFTSYAMSWVRQAPGKGLEWVSGI |
| | DGSGGGTNYADSVKGRFTISRDNSKNTLYLQMNSLRAEDTAVYYCARAYYDILTGYPVDGM |
| | DVWGQGTTVTVSS [SEQ ID NO: 17] |
| DNA | GACATCGTGATGACCCAGTCTCCAGACTCCCTGGCTGTGTCTCTGGGCGAGCGTGCCACCA |
| for | TCAACTGCAAGTCCAGCCAGAGTGTTTTAAGCAGCTATAACAATGAGAACAATTTAGCTTG |
| V_{L}-V_{H} | GTATCAGCAGAAACCAGGACAGCCTCCTAAGCTGCTCATTTACGCCGCATCTACCCGGGAA |
| scFv | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATTATAGCGAACCTTA |
| | TACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTACCAG |
| | CTATGCCATGAGCTGGGTCCGCCAGGCTCCAGGGAAGGGGCTGGAGTGGGTGTCAGGCATT |
| | GACGGTAGCGGTGGTGGCACAAATTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGAGAGCGTATTACGATATTTTGACTGGTTACCCCGTGGACGGTATG |
| | GACGTCTGGGGCCAAGGGACCACGGTCACCGTCTCCTCA [SEQ ID NO: 23] |
| V_{H}-V_{L} | EVQLLESGGGLVQPGGSLRLSCAASGFTFTSYAMSWVRQAPGKGLEWVSGIDGSGGGTNYA |
| scFv | DSVKGRFTISRDNSKNTLYLQMNSLRAEDTAVYYCARAYYDILTGYPVDGMDVWGQGTTVT |
| | VSSGGGGSGGGGSGGGSGGGGSDIVMTQSPDSLAVSLGERATINCKSSQSVLSSYNNENNL |
| | AWYQQKPGQPPKLLIYAASTRESGVPDRFSGSGSGTDFTLTISSLQAEDVAVYYCQQYYSE |
| | PYTFGQGTKVEIK [SEQ ID NO: 69] |

In certain embodiments, the anti-CD371 scFv comprises a V_{H} comprising the amino acid sequence set forth in SEQ ID NO: 5 and a V_{H} comprising the amino acid sequence set forth in SEQ ID NO: 6, optionally with a linker sequence, for example a linker peptide, between the heavy chain variable region and the light chain variable region. In certain embodiments, the linker comprises the amino acid sequence set forth in SEQ ID NO: 13. SEQ ID NOs: 5 and 6 are provided in Table 3 below. In certain embodiments, the anti-CD371 scFv is designated as “B031_P1_PH1D6” (also referred to as “D6”).

In certain embodiments, the anti-CD371 scFv is a scFv-Fc fusion protein or full-length human IgG with V_{H} and V_{L} regions or CDRs selected from Table 3. In certain embodiments, the anti-CD371 scFv comprises a V_{H} comprising the amino acid sequence set forth in SEQ ID NO: 5. In certain embodiments, the anti-CD371 scFv comprises a V_{L} comprising the amino acid sequence set forth in SEQ ID NO: 6.

In certain embodiments, the anti-CD371 scFv comprises a V_{H} comprising the amino acid sequence set forth in SEQ ID NO: 5 and a V_{L} comprising the amino acid sequence set forth in SEQ ID NO: 6.

In certain embodiments, the anti-CD371 scFv comprises a V_{H} CDR1 comprising the amino acid sequence set forth in SEQ ID NO: 40 or a conservative modification thereof, a V_{H} CDR2 comprising the amino acid sequence set forth in SEQ ID NO: 41 or a conservative modification thereof, and a V_{H} CDR3 comprising the amino acid sequence set forth in SEQ ID NO: 42 or a conservative modification thereof. SEQ ID NOs: 40-42 are provided in Table 3.

In certain embodiments, the anti-CD371 scFv comprises a V_{L} CDR1 comprising the amino acid sequence set forth in SEQ ID NO: 43 or a conservative modification thereof, a V_{L} CDR2 comprising the amino acid sequence set forth in SEQ ID NO: 44 or a conservative modification thereof, and a V_{L} CDR3 comprising the amino acid sequence set forth in SEQ ID NO: 45 or a conservative modification thereof. SEQ ID NOs: 43-45 are provided in Table 3.

In certain embodiments, the anti-CD371 scFv comprises a V_{H} CDR1 comprising the amino acid sequence set forth in SEQ ID NO: 40 or a conservative modification thereof, a V_{H} CDR2 comprising the amino acid sequence set forth in SEQ ID NO: 41 or a conservative modification thereof, a V_{H} CDR3 comprising the amino acid sequence set forth in SEQ ID NO: 42 or a conservative modification thereof, a V_{L} CDR1 comprising the amino acid sequence set forth in SEQ ID NO: 43 or a conservative modification thereof, a V_{L} CDR2 comprising the amino acid sequence set forth in SEQ ID NO: 44 or a conservative modification thereof, and a V_{L} CDR3 comprising the amino acid sequence set forth in SEQ ID NO: 45 or a conservative modification thereof.

In certain embodiments, the anti-CD371 scFv comprises a V_{H} CDR1 comprising the amino acid sequence set forth in SEQ ID NO: 40, a V_{H} CDR2 comprising the amino acid sequence set forth in SEQ ID NO: 41, a V_{H} CDR3 comprising the amino acid sequence set forth in SEQ ID NO: 42, a V_{L} CDR1 comprising the amino acid sequence set forth in SEQ ID NO: 43, a V_{L} CDR2 comprising the amino acid sequence set forth in SEQ ID NO: 44, and a V_{L} CDR3 comprising the amino acid sequence set forth in SEQ ID NO: 45.

In certain embodiments, the anti-CD371 scFv comprises a V_{H} comprising the amino acid sequence set forth in SEQ ID NO: 5, and a V_{L} comprising the amino acid sequence set forth in SEQ ID NO: 6. In certain embodiments, the V_{H} and V_{L} are linked via a linker. In certain embodiments, the linker comprises the amino acid sequence set forth in SEQ ID NO: 13.

In certain embodiments, a heavy chain variable region (V_{H}) is positioned at the N-terminus. In certain embodiments, the variable regions are positioned from the N- to the C-terminus: V_{H}-V_{L}. In certain embodiments, the anti-CD371 scFv comprises the amino acid sequence set forth in SEQ ID NO: 70, which is provided in Table 3.

In certain embodiments, a light chain variable region (V_{L}) is positioned at the N-terminus. In certain embodiments, the variable regions are positioned from the N- to the C-terminus: V_{L}-V_{H}. In certain embodiments, anti-CD371 scFv comprises the amino acid sequence set forth in SEQ ID NO: 18. An exemplary nucleotide sequence encoding the amino acid sequence of SEQ ID NO: 18 is set forth in SEQ ID NO: 24. SEQ ID NOS: 18 and 24 are provided in Table 3 below.

TABLE 3

| | CDRs | | |
|---|---|---|---|
| | 1 | 2 | 3 |
| V_{H} | GFTFTDYA [SEQ ID NO: | IDGSGGST [SEQ ID | ALELGATTVY [SEQ ID |
| | 40] | NO: 41] | NO: 42] |
| V_{L} | QSVLRSSNNKNN [SEQ | AAS [SEQ ID | QQYYREPLT [SEQ ID |
| | ID NO: 43] | NO: 44] | NO: 45] |

| | |
|---|---|
| Full | EVQLLESGGGLVQPGGSLRLSCAASGFTFTDYAMSWVRQAPGKGLEWVSDIDGSGGSTDYA |
| V_{H} | DSVKGRFTISRDNSKNTLYLQMNSLRAEDTAVYYCALELGATTVYWGQGTLVTVSS [SEQ |
| | ID NO: 5] |
| Full | DIVMTQSPDSLAVSLGERATINCKSSQSVLRSSNNKNNLAWYQQKPGQPPKLLIYAASTRE |
| V_{L} | SGVPDRFSGSGSGTDFTLTISSLQAEDVAVYYCQQYYREPLTFGQGTKVEIK [SEQ ID |
| | NO: 6] |
| V_{L}-V_{H} | DIVMTQSPDSLAVSLGERATINCKSSQSVLRSSNNKNNLAWYQQKPGQPPKLLIYAASTRE |
| scFv | SGVPDRFSGSGSGTDFTLTISSLQAEDVAVYYCQQYYREPLTFGQGTKVEIKGGGGSGGGG |
| | SGGGSGGGGSEVQLLESGGGLVQPGGSLRLSCAASGFTFTDYAMSWVRQAPGKGLEWVSDI |
| | DGSGGSTDYADSVKGRFTISRDNSKNTLYLQMNSLRAEDTAVYYCALELGATTVYWGQGTL |
| | VTVSS [SEQ ID NO: 18] |
| DNA | GACATCGTGATGACCCAGTCTCCAGACTCCCTGGCTGTGTCTCTGGGCGAGCGTGCCACCA |
| for | TCAACTGCAAGTCCAGCCAGAGTGTTTTACGCAGCAGCAACAATAAAAACAATTTAGCTTG |
| V_{L}-V_{H} | GTATCAGCAGAAACCAGGACAGCCTCCTAAGCTGCTCATTTACGCCGCATCTACCCGGGAA |
| scFv | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATTATCGCGAACCTCT |
| | GACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTACCGA |
| | CTATGCCATGAGCTGGGTCCGCCAGGCTCCAGGGAAGGGGCTGGAGTGGGTGTCAGACATT |
| | GACGGTAGCGGTGGTAGCACAGACTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGCTAGAGCTGGGAGCTACTACCGTCTACTGGGGCCAGGGAACCCTG |
| | GTCACCGTCTCCTCA [SEQ ID NO: 24] |
| V_{H}-V_{L} | EVQLLESGGGLVQPGGSLRLSCAASGFTFTDYAMSWVRQAPGKGLEWVSDIDGSGGSTDYA |
| scFv | DSVKGRFTISRDNSKNTLYLQMNSLRAEDTAVYYCALELGATTVYWGQGTLVTVSSGGGGS |
| | GGGGSGGGSGGGGSDIVMTQSPDSLAVSLGERATINCKSSQSVLRSSNNKNNLAWYQQKPG |
| | QPPKLLIYAASTRESGVPDRFSGSGSGTDFTLTISSLQAEDVAVYYCQQYYREPLTFGQGT |
| | KVEIK [SEQ ID NO: 70] |

In certain embodiments, the anti-CD371 scFv comprises a V_{H} comprising the amino acid sequence set forth in SEQ ID NO: 7 and a V_{L} comprising the amino acid sequence set forth in SEQ ID NO: 8, optionally with a linker sequence, for example a linker peptide, between the heavy chain variable region and the light chain variable region. In certain embodiments, the linker comprises the amino acid sequence set forth in SEQ ID NO: 13. SEQ ID NOs: 7 and 8 are provided in Table 4 below. In certain embodiments, the anti-CD371 scFv is designated as “B031_P1_PH2A11” (also referred to as “A11”).

In certain embodiments, the anti-CD371 scFv is a scFv-Fc fusion protein or full length human IgG with V_{H} and V_{L} regions or CDRs selected from Table 4. In certain embodiments, the anti-CD371 scFv comprises a V_{H} comprising the amino acid sequence set forth in SEQ ID NO: 13 In certain embodiments, the anti-CD371 scFv comprises a V_{L} comprising the amino acid sequence set forth in SEQ ID NO: 14. In certain embodiments, the anti-CD371 scFv comprises a V_{H} comprising the amino acid sequence set forth in SEQ ID NO: 7 and a V_{L} comprising the amino acid sequence set forth in SEQ ID NO: 8. SEQ ID NOs: 7 and 8 are provided in Table 4.

In certain embodiments, the anti-CD371 scFv comprises a V_{H} CDR1 comprising the amino acid sequence set forth in SEQ ID NO: 46 or a conservative modification thereof, a V_{H} CDR2 comprising the amino acid sequence set forth in SEQ ID NO:47 or a conservative modification thereof, and a V_{H} CDR3 comprising the amino acid sequence set forth in SEQ ID NO: 48 or a conservative modification thereof. SEQ ID NOs: 46-48 are provided in Table 4.

In certain embodiments, the anti-CD371 scFv comprises a V_{L} CDR1 comprising the amino acid sequence set forth in SEQ ID NO: 49 or a conservative modification thereof, a V_{L} CDR2 comprising the amino acid sequence set forth in SEQ ID NO: 50 or a conservative modification thereof, and a V_{L} CDR3 comprising the amino acid sequence set forth in SEQ ID NO: 51 or a conservative modification thereof. SEQ ID NOs: 49-51 are provided in Table 4.

In certain embodiments, the anti-CD371 scFv comprises a V_{H} CDR1 comprising the amino acid sequence set forth in SEQ ID NO: 46 or a conservative modification thereof, a V_{H} CDR2 comprising the amino acid sequence set forth in SEQ ID NO: 47 or a conservative modification thereof, a V_{H} CDR3 comprising the amino acid sequence set forth in SEQ ID NO: 48 or a conservative modification thereof, a V_{L} CDR1 comprising the amino acid sequence set forth in SEQ ID NO: 49 or a conservative modification thereof, a V_{L} CDR2 comprising the amino acid sequence set forth in SEQ ID NO: 50 or a conservative modification thereof, and a V_{L} CDR3 comprising the amino acid sequence set forth in SEQ ID NO: 51 or a conservative modification thereof.

In certain embodiments, the anti-CD371 scFv comprises a V_{H} CDR1 comprising the amino acid sequence set forth in SEQ ID NO: 46, a V_{H} CDR2 comprising the amino acid sequence set forth in SEQ ID NO: 47, a V_{H} CDR3 comprising the amino acid sequence set forth in SEQ ID NO: 48, a V_{L} CDR1 comprising the amino acid sequence set forth in SEQ ID NO: 49, a V_{L} CDR2 comprising the amino acid sequence set forth in SEQ ID NO: 50, and a V_{L} CDR3 comprising the amino acid sequence set forth in SEQ ID NO: 51.

In certain embodiments, the anti-CD371 scFv comprises a V_{H} comprising the amino acid sequence set forth in SEQ ID NO: 7, and a V_{L} comprising the amino acid sequence set forth in SEQ ID NO: 8. In certain embodiments, the V_{H} and V_{L} are linked via a linker. In certain embodiments, the linker comprises the amino acid sequence set forth in SEQ ID NO: 13.

In certain embodiments, a heavy chain variable region (V_{H}) is positioned at the N-terminus. In certain embodiments, the variable regions are positioned from the N- to the C-terminus: V_{H}-V_{L}. In certain embodiments, the CD371 scFv comprises the amino acid sequence set forth in SEQ ID NO: 71, which is provided in Table 4.

In certain embodiments, a light chain variable region (V_{L}) is positioned at the N-terminus. In certain embodiments, the variable regions are positioned from the N- to the C-terminus: V_{L}-V_{H}. In certain embodiments, scFv comprises the amino acid sequence set forth in SEQ ID NO: 19. An exemplary nucleotide sequence encoding the amino acid sequence of SEQ ID NO: 19 is set forth in SEQ ID NO: 25. SEQ ID NOS: 19 and 25 are provided in Table 4 below.

TABLE 4

| | CDRs | | |
|---|---|---|---|
| | 1 | 2 | 3 |
| V_{H} | GFTFTSTQ [SEQ | ISGYGGST [SEQ ID | AKDTEVSGDAFDI [SEQ ID |
| | ID NO: 46] | NO: 47] | NO: 48] |
| V_{L} | QSVDSSN [SEQ ID | GAS [SEQ ID NO: 50] | QQYRSWPIT [SEQ ID NO: |
| | NO: 49] | | 51] |

| | |
|---|---|
| Full | EVQLLESGGGLVQPGGSLRLSCAASGFTFTSTQMSWVRQAPGKGLEWVSEISGYGGSTYYA |
| V_{H} | DSVKGRFTISRDNSKNTLYLQMNSLRAEDTAVYYCAKDTEVSGDAFDIWGQGTMVTVSS |
| | [SEQ ID NO: 7] |
| Full | EIVLTQSPGTLSLSPGERATLSCRASQSVDSSNLAWYQQKPGQAPRLLIYGASSRATGIPD |
| V_{L} | RFSGSGSGTDFTLTISRLEPEDFAVYYCQQYRSWPITFGQGTKVEIK [SEQ ID |
| | NO: 8] |
| V_{L}-V_{H} | EIVLTQSPGTLSLSPGERATLSCRASQSVDSSNLAWYQQKPGQAPRLLIYGASSRATGIPD |
| scFv | RFSGSGSGTDFTLTISRLEPEDFAVYYCQQYRSWPITFGQGTKVEIKGGGGSGGGGSGGGS |
| | GGGGSEVQLLESGGGLVQPGGSLRLSCAASGFTFTSTQMSWVRQAPGKGLEWVSEISGYGG |
| | STYYADSVKGRFTISRDNSKNTLYLQMNSLRAEDTAVYYCAKDTEVSGDAFDIWGQGTMVT |
| | VSS [SEQ ID NO: 19] |
| DNA | GAAATTGTGTTGACGCAGTCTCCAGGCACCCTGTCTTTGTCTCCAGGGGAACGTGCCACCC |
| for | TCTCCTGCCGTGCCAGTCAGAGTGTTGACAGCAGCAATTTAGCCTGGTATCAGCAGAAACC |
| V_{L}-V_{H} | TGGCCAGGCTCCCCGACTCCTCATCTATGGCGCATCTAGCCGTGCCACTGGTATCCCAGAC |
| scFv | CGTTTCAGTGGCAGTGGGTCTGGGACAGACTTCACTCTCACCATCAGCAGACTGGAGCCTG |
| | AAGATTTTGCAGTGTATTACTGTCAGCAGTATCGCAGCTGGCCTATCACGTTCGGCCAAGG |
| | TACCAAGGTGGAAATCAAAGGTGGTGGTGGTTCAGGTGGTGGTGGTTCTGGCGGCGGCTCC |
| | GGTGGTGGTGGATCCGAGGTGCAGCTGTTGGAGTCTGGGGGAGGCTTGGTACAGCCTGGGG |
| | GGTCCCTGCGACTCTCCTGTGCAGCCTCTGGATTCACCTTTACCAGCACCCAGATGAGCTG |
| | GGTCCGCCAGGCTCCAGGGAAGGGGCTGGAGTGGGTGTCAGAGATTAGCGGTTATGGTGGT |
| | AGCACATACTACGCAGACTCCGTGAAGGGCCGGTTCACCATCTCCCGTGACAATTCCAAGA |
| | ACACGCTGTATCTGCAAATGAACAGCCTGCGTGCCGAGGACACGGCTGTGTATTACTGTGC |
| | AAAAGACACGGAGGTTTCGGGAGATGCTTTTGATATCTGGGGCCAAGGGACAATGGTCACC |
| | GTCTCTTCA [SEQ ID NO: 25] |
| V_{H}-V_{L} | EVQLLESGGGLVQPGGSLRLSCAASGFTFTSTQMSWVRQAPGKGLEWVSEISGYGGSTYYA |
| scFv | DSVKGRFTISRDNSKNTLYLQMNSLRAEDTAVYYCAKDTEVSGDAFDIWGQGTMVTVSSGG |
| | GGSGGGGSGGGSGGGGSEIVLTQSPGTLSLSPGERATLSCRASQSVDSSNLAWYQQKPGQA |
| | PRLLIYGASSRATGIPDRFSGSGSGTDFTLTISRLEPEDFAVYYCQQYRSWPITFGQGTKV |
| | EIK [SEQ ID NO: 71] |

In certain embodiments, the anti-CD371 scFv comprises a V_{H} comprising the amino acid sequence set forth in SEQ ID NO: 9 and a V_{L} comprising the amino acid sequence set forth in SEQ ID NO: 10, optionally with a linker sequence, for example a linker peptide, between the heavy chain variable region and the light chain variable region. In certain embodiments, the linker comprises the amino acid sequence set forth in SEQ ID NO: 13. SEQ ID NOs: 9 and 10 are provided Table 5. In certain embodiments, the anti-CD371 scFv is designated as “B031_P1_PH2E4” (also referred to as “E4”).

In certain embodiments, the anti-CD371 scFv is a scFv-Fc fusion protein or a full-length human IgG with V_{H} and V_{L} regions or CDRs selected from Table 5. In certain embodiments, the anti-CD371 scFv comprises a V_{H} comprising the amino acid sequence set forth in SEQ ID NO: 9. In certain embodiments, the anti-CD371 scFv comprises a V_{L} comprising the amino acid sequence set forth in SEQ ID NO: 10. In certain embodiments, the anti-CD371 scFv comprises a V_{H} comprising the amino acid sequence set forth in SEQ ID NO: 9 and a V_{L} comprising the amino acid sequence set forth in SEQ ID NO: 10.

In certain embodiments, the anti-CD371 scFv comprises a V_{H} CDR1 comprising the amino acid sequence set forth in SEQ ID NO: 52 or a conservative modification thereof, a V_{H} CDR2 comprising the amino acid sequence set forth in SEQ ID NO: 53 or a conservative modification thereof, and a V_{H} CDR3 comprising the amino acid sequence set forth in SEQ ID NO: 54 or a conservative modification thereof. SEQ ID NOs: 52-54 are provided in Table 5.

In certain embodiments, the anti-CD371 scFv comprises a V_{L} CDR1 comprising the amino acid sequence set forth in SEQ ID NO: 55 or a conservative modification thereof, a V_{L} CDR2 comprising the amino acid sequence set forth in SEQ ID NO: 56 or a conservative modification thereof, and a V_{L} CDR3 comprising the amino acid sequence set forth in SEQ ID NO: 57 or a conservative modification thereof. SEQ ID NOs: 55-57 are provided in Table 5. In certain embodiments, the anti-CD371 scFv comprises a V_{H} CDR1 comprising the amino acid sequence set forth in SEQ ID NO: 52 or a conservative modification thereof, a V_{H} CDR2 comprising the amino acid sequence set forth in SEQ ID NO: 53 or a conservative modification thereof, a V_{H} CDR3 comprising the amino acid sequence set forth in SEQ ID NO: 54 or a conservative modification thereof, a V_{L} CDR1 comprising the amino acid sequence set forth in SEQ ID NO: 55 or a conservative modification thereof, a V_{L} CDR2 comprising the amino acid sequence set forth in SEQ ID NO: 56 or a conservative modification thereof, and a V_{L} CDR3 comprising the amino acid sequence set forth in SEQ ID NO: 57 or a conservative modification thereof.

In certain embodiments, the anti-CD371 scFv comprises a V_{H} CDR1 comprising the amino acid sequence set forth in SEQ ID NO: 52, a V_{H} CDR2 comprising the amino acid sequence set forth in SEQ ID NO: 53, a V_{H} CDR3 comprising the amino acid sequence set forth in SEQ ID NO: 54, a V_{L} CDR1 comprising the amino acid sequence set forth in SEQ ID NO: 55, a V_{L} CDR2 comprising the amino acid sequence set forth in SEQ ID NO: 56, and a V_{L} CDR3 comprising the amino acid sequence set forth in SEQ ID NO: 57.

In certain embodiments, the anti-CD371 scFv comprises a V_{H} comprising the amino acid sequence set forth in SEQ ID NO: 9, and a V_{L} comprising the amino acid sequence set forth in SEQ ID NO: 10. In certain embodiments, the V_{H} and V_{L} are linked via a linker. In certain embodiments, the linker comprises the amino acid sequence set forth in SEQ ID NO: 13.

In certain embodiments, a heavy chain variable region (V_{H}) is positioned at the N-terminus. In certain embodiments, the variable regions are positioned from the N- to the C-terminus: V_{H}-V_{L}. In certain embodiments, the anti-CD371 scFv comprises the amino acid sequence set forth in SEQ ID NO: 72, which is provided in Table 5.

In certain embodiments, a light chain variable region (V_{L}) is positioned at the N-terminus. In certain embodiments, the variable regions are positioned from the N- to the C-terminus: V_{L}-V_{H}. In certain embodiments, the anti-CD371 scFv comprises the amino acid sequence set forth in SEQ ID NO: 20. An exemplary nucleotide sequence encoding the amino acid sequence of SEQ ID NO: 20 is set forth in SEQ ID NO: 26. SEQ ID NOS: 20 and 26 are provided in Table 5 below.

TABLE 5

| | CDRs | | |
|---|---|---|---|
| | 1 | 2 | 3 |
| V_{H} | GFTFTSYY [SEQ ID | ISGSGDST [SEQ ID | AREAGGDYDSGAFDI [SEQ |
| | NO: 52] | NO: 53] | ID NO: 54] |
| V_{L} | QSVLYSGNNKNY [SEQ | GAS [SEQ ID NO: | QQYDYAPFT [SEQ ID |
| | ID NO: 55] | 56] NO: | 57] |

| | |
|---|---|
| Full | EVQLLESGGGLVQPGGSLRLSCAASGFTFTSYYMSWVRQAPGKGLEWVSGISGSGDSTSYA |
| V_{H} | DSVKGRFTISRDNSKNTLYLQMNSLRAEDTAVYYCAREAGGDYDSGAFDIWGQGTMVTVSS |
| | [SEQ ID NO: 9] |
| Full | DIVMTQSPDSLAVSLGERATINCKSSQSVLYSGNNKNYLAWYQQKPGQPPKLLIYGASTRE |
| V_{L} | SGVPDRFSGSGSGTDFTLTISSLQAEDVAVYYCQQYDYAPFTFGQGTKVEIK [SEQ ID |
| | NO: 10] |
| V_{L}-V_{H} | DIVMTQSPDSLAVSLGERATINCKSSQSVLYSGNNKNYLAWYQQKPGQPPKLLIYGASTRE |
| scFv | SGVPDRFSGSGSGTDFTLTISSLQAEDVAVYYCQQYDYAPFTFGQGTKVEIKGGGGSGGGG |
| | SGGGSGGGGSEVQLLESGGGLVQPGGSLRLSCAASGFTFTSYYMSWVRQAPGKGLEWVSGI |
| | SGSGDSTSYADSVKGRFTISRDNSKNTLYLQMNSLRAEDTAVYYCAREAGGDYDSGAFDIW |
| | GQGTMVTVSS [SEQ ID NO: 20] |
| DNA | GACATCGTGATGACCCAGTCTCCAGACTCCCTGGCTGTGTCTCTGGGCGAGCGTGCCACCA |
| for | TCAACTGCAAGTCCAGCCAGAGTGTTTTATATAGCGGCAACAATAAAAACTATTTAGCTTG |
| V_{L}-V_{H} | GTATCAGCAGAAACCAGGACAGCCTCCTAAGCTGCTCATTTACGGCGCATCTACCCGGGAA |
| scFv | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAATATGACTATGCCCCTTT |
| | TACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTACCAG |
| | CTATTATATGAGCTGGGTCCGCCAGGCTCCAGGGAAGGGGCTGGAGTGGGTGTCAGGCATT |
| | AGCGGTAGCGGTGACAGCACAAGCTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | TGTGTATTACTGTGCGAGAGAGGCAGGTGGTGACTACGATAGTGGTGCTTTTGATATCTGG |
| | GGCCAAGGGACAATGGTCACCGTCTCTTCA [SEQ ID NO: 26] |
| V_{H}-V_{L} | EVQLLESGGGLVQPGGSLRLSCAASGFTFTSYYMSWVRQAPGKGLEWVSGISGSGDSTSYA |
| scFv | DSVKGRFTISRDNSKNTLYLQMNSLRAEDTAVYYCAREAGGDYDSGAFDIWGQGTMVTVSS |
| | GGGGSGGGGSGGGSGGGGSDIVMTQSPDSLAVSLGERATINCKSSQSVLYSGNNKNYLAWY |
| | QQKPGQPPKLLIYGASTRESGVPDRFSGSGSGTDFTLTISSLQAEDVAVYYCQQYDYAPFT |
| | FGQGTKVEIK [SEQ ID NO: 72] |

In certain embodiments, the anti-CD371 scFv comprises a V_{H} comprising the amino acid sequence set forth in SEQ ID NO: 11 and a V_{L} comprising the amino acid sequence set forth in SEQ ID NO: 12, optionally with a linker sequence, for example a linker peptide, between the heavy chain variable region and the light chain variable region. In certain embodiments, the linker comprises the amino acid sequence set forth in SEQ ID NO: 13. SEQ ID NOs: 11 and 12 are provided in Table 6 below. In certain embodiments, the anti-CD371 scFv is designated as “B031_P1_PH2E8” (also referred to as “E8”).

In certain embodiments, the anti-CD371 scFv is a scFv-Fc fusion protein or a full-length human IgG with V_{H} and V_{L} regions or CDRs selected from Table 6. In certain embodiments, the anti-CD371 scFv comprises a V_{H} comprising the amino acid sequence set forth in SEQ ID NO: 11. In certain embodiments, the anti-CD371 scFv comprises a V_{L} comprising the amino acid sequence set forth in SEQ ID NO: 12. In certain embodiments, the anti-CD371 scFv comprises a V_{H} comprising the amino acid sequence set forth in SEQ ID NO: 11 and a V_{L} comprising the amino acid sequence set forth in SEQ ID NO: 12.

In certain embodiments, the anti-CD371 scFv comprises a V_{H} CDR1 comprising the amino acid sequence set forth in SEQ ID NO: 58 or a conservative modification thereof, a V_{H} CDR2 comprising the amino acid sequence set forth in SEQ ID NO: 59 or a conservative modification thereof, and a V_{H} CDR3 comprising the amino acid sequence set forth in SEQ ID NO: 60 or a conservative modification thereof. SEQ ID NOs: 58-60 are provided in Table 6.

In certain embodiments, the anti-CD371 scFv comprises a V_{L} CDR1 comprising the amino acid sequence set forth in SEQ ID NO: 61 or a conservative modification thereof, a V_{L} CDR2 comprising the amino acid sequence set forth in SEQ ID NO: 62 or a conservative modification thereof, and a V_{L} CDR3 comprising the amino acid sequence set forth in SEQ ID NO: 63 or a conservative modification thereof. SEQ ID NOs: 61-63 are provided in Table 6.

In certain embodiments, the anti-CD371 scFv comprises a V_{H} CDR1 comprising the amino acid sequence set forth in SEQ ID NO: 58 or a conservative modification thereof, a V_{H} CDR2 comprising the amino acid sequence set forth in SEQ ID NO: 59 or a conservative modification thereof, a V_{H} CDR3 comprising the amino acid sequence set forth in SEQ ID NO: 60 or a conservative modification thereof, a V_{L} CDR1 comprising the amino acid sequence set forth in SEQ ID NO: 61 or a conservative modification thereof, a V_{L} CDR2 comprising the amino acid sequence set forth in SEQ ID NO: 62 or a conservative modification thereof, and a V_{L} CDR3 comprising the amino acid sequence set forth in SEQ ID NO: 63 or a conservative modification thereof.

In certain embodiments, the anti-CD371 scFv comprises a V_{H} CDR1 comprising the amino acid sequence set forth in SEQ ID NO: 58, a V_{H} CDR2 comprising the amino acid sequence set forth in SEQ ID NO: 59, a V_{H} CDR3 comprising the amino acid sequence set forth in SEQ ID NO: 60, a V_{L} CDR1 comprising the amino acid sequence set forth in SEQ ID NO: 61, a V_{L} CDR2 comprising the amino acid sequence set forth in SEQ ID NO: 62, and a V_{L} CDR3 comprising the amino acid sequence set forth in SEQ ID NO: 63.

In certain embodiments, the anti-CD371 scFv comprises a V_{H} comprising the amino acid sequence set forth in SEQ ID NO: 11, and a V_{L} comprising the amino acid sequence set forth in SEQ ID NO: 12. In certain embodiments, the V_{H} and V_{L} are linked via a linker. In certain embodiments, the linker comprises the amino acid sequence set forth in SEQ ID NO: 13.

In certain embodiments, a heavy chain variable region (V_{H}) is positioned at the N-terminus. In certain embodiments, the variable regions are positioned from the N- to the C-terminus: V_{H}-V_{L}. In certain embodiments, the anti-CD371 scFv comprises the amino acid sequence set forth in SEQ ID NO: 73, which is provided in Table 6.

In certain embodiments, a light chain variable region (V_{L}) is positioned at the N-terminus. In certain embodiments, the variable regions are positioned from the N- to the C-terminus: V_{L}-V_{H}. In certain embodiments, the anti-CD371 scFv comprises the amino acid sequence set forth in SEQ ID NO: 21. An exemplary nucleotide sequence encoding the amino acid sequence of SEQ ID NO: 21 is set forth in SEQ ID NO: 27. SEQ ID NOS: 21 and 27 are provided in Table 6 below.

TABLE 6

| | CDRs | | |
|---|---|---|---|
| | 1 | 2 | 3 |
| V_{H} | GFTFSSYA [SEQ ID | IDGEGGYT [SEQ ID | AREGVDYDILTGYYPYGMDV |
| | NO: 58] | NO: 59] | [SEQ ID NO: 60] |
| V_{L} | QSVLDSSNNKNY [SEQ | DAS [SEQ ID NO: | QQGTSSPLT [SEQ ID |
| | ID NO: 61] | 62] | NO: 63] |

| | |
|---|---|
| Full | EVQLLESGGGLVQPGGSLRLSCAASGFTFSSYAMSWVRQAPGKGLEWVSEIDGEGGYTNYA |
| V_{H} | DSVKGRFTISRDNSKNTLYLQMNSLRAEDTAVYYCAREGVDYDILTGYYPYGMDVWGQGTT |
| | VTVSS [SEQ ID NO: 11] |
| Full | DIVMTQSPDSLAVSLGERATINCKSSQSVLDSSNNKNYLAWYQQKPGQPPKLLIYDASTRE |
| V_{L} | SGVPDRFSGSGSGTDFTLTISSLQAEDVAVYYCQQGTSSPLTFGQGTKVEIK [SEQ ID |
| | NO: 12] |
| V_{L}-V_{H} | DIVMTQSPDSLAVSLGERATINCKSSQSVLDSSNNKNYLAWYQQKPGQPPKLLIYDASTRE |
| scFv | SGVPDRFSGSGSGTDFTLTISSLQAEDVAVYYCQQGTSSPLTFGQGTKVEIKGGGGSGGGG |
| | SGGGSGGGGSEVQLLESGGGLVQPGGSLRLSCAASGFTFSSYAMSWVRQAPGKGLEWVSEI |
| | DGEGGYTNYADSVKGRFTISRDNSKNTLYLQMNSLRAEDTAVYYCAREGVDYDILTGYYPY |
| | GMDVWGQGTTVTVSS [SEQ ID NO: 21] |
| DNA | GACATCGTGATGACCCAGTCTCCAGACTCCCTGGCTGTGTCTCTGGGCGAGCGTGCCACCA |
| for | TCAACTGCAAGTCCAGCCAGAGTGTTTTAGACAGCAGCAACAATAAAAACTATTTAGCTTG |
| V_{L}-V_{H} | GTATCAGCAGAAACCAGGACAGCCTCCTAAGCTGCTCATTTACGACGCATCTACCCGGGAA |
| scFv | TCCGGGGTCCCTGACCGATTCAGTGGCAGCGGGTCTGGGACAGATTTCACTCTCACCATCA |
| | GCAGCCTGCAGGCTGAAGATGTGGCAGTTTATTACTGTCAGCAAGGCACCAGCAGCCCTCT |
| | GACGTTCGGCCAAGGTACCAAGGTGGAAATCAAAGGTGGTGGTGGTTCAGGTGGTGGTGGT |
| | TCTGGCGGCGGCTCCGGTGGTGGTGGATCCGAGGTGCAGCTGTTGGAGTCTGGGGGAGGCT |
| | TGGTACAGCCTGGGGGGTCCCTGCGACTCTCCTGTGCAGCCTCTGGATTCACCTTTAGCAG |
| | CTATGCCATGAGCTGGGTCCGCCAGGCTCCAGGGAAGGGGCTGGAGTGGGTGTCAGAGATT |
| | GACGGTGAGGGTGGTTATACAAATTACGCAGACTCCGTGAAGGGCCGGTTCACCATCTCCC |
| | GTGACAATTCCAAGAACACGCTGTATCTGCAAATGAACAGCCTGCGTGCCGAGGACACGGC |
| | CGTGTATTACTGTGCGAGAGAAGGGGTAGATTACGATATTTTGACTGGTTATTATCCTTAC |
| | GGTATGGACGTCTGGGGCCAAGGGACCACGGTCACCGTCTCCTCA [SEQ ID NO: 27] |
| V_{H}-V_{L} | EVQLLESGGGLVQPGGSLRLSCAASGFTFSSYAMSWVRQAPGKGLEWVSEIDGEGGYTNYA |
| scFv | DSVKGRFTISRDNSKNTLYLQMNSLRAEDTAVYYCAREGVDYDILTGYYPYGMDVWGQGTT |
| | VTVSSGGGGSGGGGSGGGSGGGGSDIVMTQSPDSLAVSLGERATINCKSSQSVLDSSNNKN |
| | YLAWYQQKPGQPPKLLIYDASTRESGVPDRFSGSGSGTDFTLTISSLQAEDVAVYYCQQGT |
| | SSPLTFGQGTKVEIK [SEQ ID NO: 73] |

### 5.3.2. Monoclonal Antibodies

The presently disclosed subject matter provides antibodies (e.g., human antibodies, e.g., human monoclonal antibodies) that specifically bind to CD371 (e.g., human CD371). The V_{H} amino acid sequences of anti-CD371 antibodies B031_P1_PH1B10 (also referred to as “B10”), B031_P1_PH1C3 (also referred to as “C3”), B031_P1_PH1D6 (also referred to as “D6”), B031_P1_PH2A11 (also referred to as “A11”), B031_P1_PH2E4 (also referred to as “E4”), and B031_P1_PH2E8 (also referred to as “E8”), are shown in SEQ ID NOs: 1, 3, 5, 7, 9, and 11, respectively. The V_{L} amino acid sequences of B10, C3, D6, A11, E4, and E8 are shown in SEQ ID NOs: 2, 4, 6, 8, 10, and 12, respectively.

Given that each of B031_P1_PH1B10 (B10), B031_P1_PH1C3 (C3), B031_P1_PH1D6 (D6), B031_P1_PH2A11 (A11), B031_P1_PH2E4 (E4), and B031_P1_PH2E8 (E8) antibodies can bind to CD371, the V_{H} and V sequences can be “mixed and matched” to create other anti-CD371 binding molecules. CD371 binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}NL pairing is replaced with a structurally similar V_{L} sequence.

In certain embodiments, the presently disclosed subject matter provides an antibody or an antigen-binding fragment or portion thereof comprising: (a) a heavy chain variable region (V_{H}) comprising an amino acid sequence selected from SEQ ID NOs: 1, 3, 5, 7, 9, and 11; and (b) a light chain variable region (V_{L}) comprising an amino acid sequence selected from SEQ ID NOs: 2, 4, 6, 8, 10, and 12; wherein the antibody or antigen-binding fragment specifically binds to CD371, e.g., human CD371. In certain embodiments, the V_{H} and V_{L} are selected from:

(a) a heavy chain variable region comprising the amino acid sequence set forth in SEQ ID NO: 1, and a light chain variable region comprising the amino acid sequence set forth in SEQ ID NO: 2; or

(b) a heavy chain variable region comprising the amino acid sequence set forth in SEQ ID NO: 3, and a light chain variable region comprising the amino acid sequence set forth in SEQ ID NO: 4;

(c) a heavy chain variable region comprising the amino acid sequence set forth in SEQ ID NO: 5, and a light chain variable region comprising the amino acid sequence set forth in SEQ ID NO: 6;

(d) a heavy chain variable region comprising the amino acid sequence set forth in SEQ ID NO: 7, and a light chain variable region comprising the amino acid sequence set forth in SEQ ID NO: 8;

(e) a heavy chain variable region comprising the amino acid sequence set forth in SEQ ID NO: 9, and a light chain variable region comprising amino acids having a sequence set forth in SEQ ID NO: 10; and

(f) a heavy chain variable region comprising the amino acid sequence set forth in SEQ ID NO: 11, and a light chain variable region comprising amino acids having a sequence set forth in SEQ ID NO: 12.

In certain embodiments, the presently disclosed subject matter provides antibodies or antigen-binding fragments thereof that comprise the heavy chain and light chain CDR1s, CDR2s and CDR3s of B10, C3, D6, A11, E4, and E8.

The amino acid sequences of the V_{H} CDR1s of B10, C3, D6, A11, E4, and E8 are shown in SEQ ID NOs: 28, 34, 40, 46, 52, and 58, respectively. The amino acid sequences of the V_{H} CDR2s of B10, C3, D6, A11, E4, and E8 antibodies are shown in SEQ ID NOs: 29, 35, 41, 47, 53, and 59, respectively. The amino acid sequences of the V_{H} CDR3s of B10, C3, D6, A11, E4, and E8 are shown in SEQ ID NOs: 30, 36, 42, 48, 54, and 60, respectively.

The amino acid sequences of the V_{L} CDR1s of B10, C3, D6, A11, E4, and E8 are shown in SEQ ID NOs: 31, 37, 43, 49, 55, and 61, respectively. The amino acid sequences of the V_{L} CDR2s of B10, C3, D6, A11, E4, and E8 are shown in SEQ ID NOs: 32, 38, 44, 50, 56, and 62, respectively. The amino acid sequences of the V_{L} CDR3s of B10, C3, D6, A11, E4, and E8 are shown in SEQ ID NOs: 33, 39, 45, 51, 57, and 63, respectively. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to CD371 and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other anti-CD371 binding molecules. CD371 binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein B10, C3, D6, A11, E4, and E8.

In certain embodiments, the presently disclosed subject matter provides an antibody or an antigen-binding fragment or portion thereof comprising:

(a) a heavy chain variable region CDR1 comprising an amino acid sequence selected from SEQ ID NOs: 28, 34, 40, 46, 52, and 58;

(b) a heavy chain variable region CDR2 comprising an amino acid sequence selected from SEQ ID NOs: 29, 35, 41, 47, 53, and 59;

(c) a heavy chain variable region CDR3 comprising an amino acid sequence selected from SEQ ID NOs: 30, 36, 42, 48, 54, and 60;

(d) a light chain variable region CDR1 comprising an amino acid sequence selected from SEQ ID NOs: 31, 37, 43, 49, 55, and 61;

(e) a light chain variable region CDR2 comprising an amino acid sequence selected from SEQ ID NOs: 32, 38, 44, 50, 56, and 62; and

(f) a light chain variable region CDR3 comprising an amino acid sequence selected from SEQ ID NOs: 33, 39, 45, 51, 57, and 63.

In certain embodiments, the antibody or antigen-binding fragment thereof comprises:

(a) a heavy chain variable region CDR1 comprising the amino acid sequence set forth in SEQ ID NO: 28;

(b) a heavy chain variable region CDR2 comprising the amino acid sequence set forth in SEQ ID NO:30 SEQ ID NO: 29;

(c) a heavy chain variable region CDR3 comprising the amino acid sequence set forth in SEQ ID NO: 30;

(d) a light chain variable region CDR1 comprising the amino acid sequence set forth in SEQ ID NO: 31;

(e) a light chain variable region CDR2 comprising the amino acid sequence set forth in SEQ ID NO:33 SEQ ID NO:33 SEQ ID NO: 32; and

(f) a light chain variable region CDR3 comprising the amino acid sequence set forth in SEQ ID NO: 33.

In certain embodiments, the antibody or antigen-binding fragment comprises:

(a) a heavy chain variable region CDR1 comprising the amino acid sequence set forth in SEQ ID NO: 34;

(b) a heavy chain variable region CDR2 comprising the amino acid sequence set forth in SEQ ID NO: 35;

(c) a heavy chain variable region CDR3 comprising the amino acid sequence set forth in SEQ ID NO: 36;

(d) a light chain variable region CDR1 comprising the amino acid sequence set forth in SEQ ID NO: 37;

(e) a light chain variable region CDR2 comprising the amino acid sequence set forth in SEQ ID NO: 38; and

(f) a light chain variable region CDR3 comprising the amino acid sequence set forth in SEQ ID NO: 39.

In certain embodiments, the antibody or antigen-binding fragment thereof comprises:

(a) a heavy chain variable region CDR1 comprising the amino acid sequence set forth in SEQ ID NO: 40;

(b) a heavy chain variable region CDR2 comprising the amino acid sequence set forth in SEQ ID NO: 41;

(c) a heavy chain variable region CDR3 comprising the amino acid sequence set forth in SEQ ID NO: 42;

(d) a light chain variable region CDR1 comprising the amino acid sequence set forth in SEQ ID NO: 43;

(e) a light chain variable region CDR2 comprising the amino acid sequence set forth in SEQ ID NO: 44; and

(f) a light chain variable region CDR3 comprising the amino acid sequence set forth in SEQ ID NO: 45.

In certain embodiments, the antibody or antigen-binding fragment thereof comprises:

(a) a heavy chain variable region CDR1 comprising the amino acid sequence set forth in SEQ ID NO: 46;

(b) a heavy chain variable region CDR2 comprising the amino acid sequence set forth in SEQ ID NO:47;

(c) a heavy chain variable region CDR3 comprising the amino acid sequence set forth in SEQ ID NO: 48;

(d) a light chain variable region CDR1 comprising the amino acid sequence set forth in SEQ ID NO: 49;

(e) a light chain variable region CDR2 comprising the amino acid sequence set forth in SEQ ID NO: 50; and

(f) a light chain variable region CDR3 comprising the amino acid sequence set forth in SEQ ID NO: 51.

In certain embodiments, the antibody or antigen-binding fragment thereof comprises:

(a) a heavy chain variable region CDR1 comprising the amino acid sequence set forth in SEQ ID NO: 52;

(b) a heavy chain variable region CDR2 comprising the amino acid sequence set forth in SEQ ID NO: 53;

(c) a heavy chain variable region CDR3 comprising the amino acid sequence set forth in SEQ ID NO: 54;

(d) a light chain variable region CDR1 comprising the amino acid sequence set forth in SEQ ID NO: 55;

(e) a light chain variable region CDR2 comprising the amino acid sequence set forth in SEQ ID NO: 56; and

(f) a light chain variable region CDR3 comprising the amino acid sequence set forth in SEQ ID NO: 57.

In certain embodiments, the antibody or antigen-binding fragment thereof comprises:

(a) a heavy chain variable region CDR1 comprising the amino acid sequence set forth in SEQ ID NO: 58;

(b) a heavy chain variable region CDR2 comprising the amino acid sequence set forth in SEQ ID NO: 59;

(c) a heavy chain variable region CDR3 comprising the amino acid sequence set forth in SEQ ID NO: 60;

(d) a light chain variable region CDR1 comprising the amino acid sequence set forth in SEQ ID NO: 61;

(e) a light chain variable region CDR2 comprising the amino acid sequence set forth in SEQ ID NO: 62; and

(f) a light chain variable region CDR3 comprising the amino acid sequence set forth in SEQ ID NO: 63.

The constant region/framework region of the anti-CD371 antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-CD371 antibody is a fully-human antibody, e.g., any one of B10, C3, D6, A11, E4, and E8. Fully-human mAbs, when administered to humans, causing serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFV) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human CD371 polypeptide (e.g., a polypeptide having the amino acid sequence set forth in SEQ ID NO:23) for cancer therapy, e.g., for treating AML.

### 5.3.3. Homologous Antibodies

In certain embodiments, a presently disclosed antibody or antigen-binding fragment thereof comprises heavy and light chain variable regions comprising amino acid sequences that are homologous or identical to the amino acid sequences of the antibodies described herein (e.g., B10, C3, D6, A11, E4, and E8 antibodies), and wherein the antibodies or antigen-binding fragments thereof retain the desired functional properties of the anti-CD371 antibodies or antigen-binding fragments thereof of the presently disclosed subject matter.

For example, the presently disclosed subject matter provides an antibody or an antigen-binding fragment or portion thereof, comprising a heavy chain variable region and a light chain variable region, wherein:

(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 1, SEQ ID NO: 3, SEQ ID NO: 5, SEQ ID NO: 7, SEQ ID NO: 9, or SEQ ID NO: 11;

(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 2, SEQ ID NO: 4, SEQ ID NO: 6, SEQ ID NO: 8, SEQ ID NO: 10, or SEQ ID NO: 12; and

wherein the antibody or antigen-binding fragment thereof specifically binds to human CD371 with a K_{d} of 1×10⁻⁷ M or less or a K_{d} of 1×10⁻⁸ M or less.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions x 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used. ( ).

### 5.3.4. Antibodies with Conservative Modifications

In certain embodiments, a presently disclosed antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., B10, C3, D6, A11, E4, and E8 antibodies), or a conservative modification thereof, and wherein the antibodies retain the desired functional properties of the anti-CD371 antibodies or antigen-binding fragments thereof of the presently disclosed subject matter. The presently disclosed subject matter provides an antibody or an antigen-binding fragment or portion thereof, comprising a heavy chain variable region comprising CDR1, CDR2, and CDR3 sequences and a light chain variable region comprising CDR1, CDR2, and CDR3 sequences, wherein:

(a) the heavy chain variable region CDR3 sequence comprises an amino acid sequence selected from the amino acid sequences of SEQ ID NOs: 30, 36, 42, 48, 54, and 60, and conservative modifications thereof;

(b) the light chain variable region CDR3 sequence comprises an amino acid sequence selected from the amino acid sequence of SEQ ID NOs: 33, 39, 45, 51, 57, and 63, and conservative modifications thereof, and

wherein the antibody or antigen-binding fragment thereof binds to human CD371 with a K_{d} of 1×10⁻⁷ M or less or a K_{d} of 1×10⁻⁸ M or less.

In certain embodiments, the heavy chain variable region CDR3 sequence comprises an amino acid sequence selected from the amino acid sequences of SEQ ID NOs: 30, 36, 42, 48, 54, and 60, and conservative modifications thereof, and the light chain variable region CDR3 sequence comprises an amino acid sequence selected from the amino acid sequences of SEQ ID NOs: 33, 39, 45, 51, 57, and 63, and conservative modifications thereof.

In certain embodiments, the heavy chain variable region CDR2 sequence comprises an amino acid sequence selected from the amino acid sequences of SEQ ID NOs: 29, 35, 41, 47, 53, and 59, and conservative modifications thereof, and the light chain variable region CDR2 sequence comprises an amino acid sequence selected from the amino acid sequences of SEQ ID NOs: 32, 38, 44, 50, 56, and 62, and conservative modifications thereof.

In certain embodiments, the heavy chain variable region CDR1 sequence comprises an amino acid sequence selected from the amino acid sequences of SEQ ID NOs: 28, 34, 40, 46, 52, and 58, and conservative modifications thereof, and the light chain variable region CDR1 sequence comprises an amino acid sequence selected from the amino acid sequences of SEQ ID NOs: 31, 37, 43, 49, 55, and 61, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 7. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 7

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

### 5.3.5. Anti-CD371 Antibodies that Cross-Compete for Binding to CD371 with Anti-CD371 Antibodies of the Invention

The presently disclosed subject matter provides antibodies or antigen-binding fragments thereof that cross-compete with any of the disclosed anti-CD371 antibodies for binding to CD371 (e.g., human CD371). For example, and not by way of limitation, the cross-competing antibodies can bind to the same epitope region, e.g., same epitope, adjacent epitope, or overlapping as any of the anti-CD371 antibodies or antigen-binding fragments thereof of the presently disclosed subject matter. In certain embodiments, the reference antibody or reference antigen-binding fragments thereof for cross-competition studies can be any one of the anti-CD371 antibodies or antigen-binding fragments thereof disclosed herein, e.g., B10, C3, D6, A11, E4, and E8 antibodies.

Such cross-competing antibodies can be identified based on their ability to cross-compete with any one of the presently disclosed anti-CD371 antibodies or antigen-binding fragments thereof in standard CD371 binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-CD371 antibodies (e.g., B10, C3, D6, A11, E4, and E8 antibodies) to CD371 (e.g., human CD371) demonstrates that the test antibody can compete with any one of the presently disclosed anti-CD371 antibodies or antigen-binding fragments thereof for binding to CD371 (e.g., human CD371) and thus binds to the same epitope region on CD371 (e.g., human CD371) as any one of the presently disclosed anti-CD371 antibodies or antigen-binding fragments thereof. In certain embodiments, the cross-competing antibody or antigen-binding fragment thereof binds to the same epitope on CD371 (e.g., human CD371) as any one of the presently disclosed anti-CD371 antibodies or antigen-binding fragments thereof.

### 5.3.6. Characterization of Antibody Binding to Antigen

Antibodies or antigen-binding fragments thereof of the presently disclosed subject can be tested for binding to CD371 by, for example, standard ELISA. To determine if the selected anti-CD371 antibodies bind to unique epitopes, each antibody can be biotinylated using commercially available reagents (Pierce, Rockford, Ill.). Competition studies using unlabeled monoclonal antibodies and biotinylated monoclonal antibodies can be performed using CD371 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-CD371 human IgGs can be further tested for reactivity with CD371 antigen by Western blotting.

In certain embodiments, the K_{d} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{d} is measured using a BIACORE® surface plasmon resonance assay. For example, an assay using a BIACORE®-2000 or a BIACORE®-3000 (BIAcore, Inc., Piscataway, N.J.)

### 5.3.7. Immunoconjugates

The presently disclosed subject provides an anti-CD371 antibody or an antigen-binding fragment thereof, conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Non-limiting Examples of cytotoxins include taxol (such as ricin, diphtheria, gelonin), cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, calecheamicin, aureastatin, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), hypomethylating agents (azacytidine and decitabine), and anti-mitotic agents (e.g., vincristine and vinblastine).

Other examples of therapeutic cytotoxins that can be conjugated to an anti-CD371 antibody disclosed herein include duocarmycins, calicheamicins, maytansines and auristatins, and derivatives thereof. Cytotoxins can be conjugated to an anti-CD371 antibody or an antigen-binding fragment thereof disclosed herein using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Anti-CD371 antibodies or antigen-binding fragments thereof of the presently disclosed subject matter also can be conjugated to a radioactive isotope to generate cytotoxic radiopharmaceuticals, also referred to as radioimmunoconjugates. Non-limiting examples of radioactive isotopes that can be conjugated to antibodies for use diagnostically or therapeutically include ⁹⁰Y, ¹³¹I, ²²⁵Ac, ²¹³Bi, ²²³Ra and ²²⁷Th. Methods for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the antibodies of the invention.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor (TNF) or interferon-γ; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 5.3.8. Bispecific Molecules

The presently disclosed subject matter provides bispecific molecules comprising an anti-CD371 antibody, or a fragment thereof, disclosed herein. A presently disclosed or an antigen-binding fragment thereof can be derivatized or linked to another functional molecule, e.g., another peptide or protein (e.g., another antibody or ligand for a receptor) to generate a bispecific molecule that binds to at least two different binding sites or target molecules. The presently disclosed or an antigen-binding fragment thereof can in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule, a presently disclosed anti-CD371 antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

The presently disclosed subject matter provides bispecific molecules comprising at least a first binding specificity for CD371 and a second binding specificity for a second target epitope. The second target epitope can be a CD371 epitope, or a non-CD371 epitope, e.g., a different antigen. In certain embodiments, the bispecific molecule is multispecific, the molecule can further include a third binding specificity. Where a first portion of a bispecific antibody binds to an antigen on a tumor cell for example and a second portion of a bispecific antibody recognizes an antigen on the surface of a human immune effector cell, the antibody is capable of recruiting the activity of that effector cell by specifically binding to the effector antigen on the human immune effector cell. In certain embodiments, bispecific antibodies, therefore, are able to form a link between effector cells, for example, T cells and tumor cells, thereby enhancing effector function. In certain embodiments, a bispecific antibody of the present disclosure comprises at least a first binding to CD371 and at least a second binding to an immune cell.

The bispecific molecules of the presently disclosed subject matter can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, Ill.).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAb×mAb, mAb×Fab, Fab×F(ab′)₂ or ligand x Fab fusion protein.

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 5.3.9. Selecting a High Affinity ScFv Against a CD371 Polypeptide

The next step is to select a phage that binds to the target antigen of interest (e.g., CD371) with a high binding affinity in a phage display library (e.g., a human phage display library) that either does not bind or that binds with a lower binding affinity. This can be accomplished by iterative binding of phage to the antigen, which is bound to a solid support, for example, beads or mammalian cells followed by removal of non-bound phage and by elution of specifically bound phage. In certain embodiments, antigens (e.g., CD371) are immobilized on a surface (e.g., a polystyrene surface). The phage library is incubated with the cells, beads or other solid support and nonbinding phage is removed by washing. Clones that bind are selected and tested.

Once selected, positive scFv clones are tested for their binding to CD371 (e.g., human CD371) on cell surfaces by flow cytometry. Briefly, phage clones are incubated with HEK293H cells over-expressing CD371. The cells are washed and then incubated with a M13 coat protein mAb. Cells are washed again and labeled with a PE-labeled anti-mouse Fab₂ prior to flow cytometry.

In other embodiments, the anti-CD371 antibodies can comprise one or more framework region amino acid substitutions designed to improve protein stability, antibody binding, expression levels or to introduce a site for conjugation of therapeutic agents. These scFv are then used to produce recombinant human monoclonal Igs in accordance with methods known to those of skill in the art.

### 5.3.10. Engineering Full Length mAb Using the Selected ScFv Fragments

Phage display technology allows for the rapid selection and production of antigen-specific scFv and Fab fragments, which are useful in and of themselves, or which can be further developed to provide complete antibodies, antigen binding proteins or antigen binding fragments thereof. Complete mAbs with Fc domains have a number of advantages over the scFv and Fab antibodies. First, only full length Abs exert immunological function such as CDC and ADCC mediated via Fc domain. Second, bivalent mAbs offer stronger antigen-binding affinity than monomeric Fab Abs. Third, plasma half-life and renal clearance will be different with the Fab and bivalent mAb. The particular features and advantages of each can be matched to the planned effector strategy. Fourth, bivalent mAb may be internalized at different rates that scFv and Fab, altering immune function or carrier function. Alpha emitters, for example, do not need to be internalized to kill the targets, but many drugs and toxins will benefit from internalization of the immune complex. In certain embodiments, therefore, once scFv clones specific for CD371 were obtained from phage display libraries, a full length IgG mAb using the scFv fragments was produced.

To produce recombinant human monoclonal IgG in Chinese hamster ovary (CHO) cells, a full length IgG mAb can be engineered based on a method known to those of skill in the art (Tomomatsu et al., Production of human monoclonal antibodies against FceRIa by a method combining in vitro immunization with phage display. Biosci Biotechnol Biochem 73(7): 1465-1469 2009). Briefly, antibody variable regions can be subcloned into mammalian expression vectors, with matching Lambda or Kappa light chain constant sequences and IgG1 subclass Fc (for example) (Lidija P, et al. An integrated vector system for the eukaryotic expression of antibodies or their fragments after selection from phage display libraries. Gene 1997; 187(1): 9-18; Lisa J H, et al. Crystallographic structure of an intact IgG1 monoclonal antibody. Journal of Molecular Biology 1998; 275 (5): 861-872). Kinetic binding analysis (Yasmina N A, et al. Probing the binding mechanism and affinity of tanezumab, a recombinant humanized anti-NGF monoclonal antibody, using a repertoire of biosensors. Protein Science 2008; 17(8): 1326-1335) can be used to confirm specific binding of full length IgG to CD371, with a K_{d} in nanomolar range.

### 5.4. Nucleic Acids Encoding the Antibodies or Antigen-Binding Fragments

The presently disclosed subject matter provides nucleic acids encoding the anti-CD371 antibodies or antigen-binding fragments thereof disclosed herein. In certain embodiments, the nucleic acid comprises or consists of the nucleotide sequence set forth in SEQ ID NO: 22, SEQ ID NO: 23, SEQ ID NO: 24, SEQ ID NO: 25, SEQ ID NO: 26, or SEQ ID NO: 27.

Furthermore provided are vectors comprising the presently disclosed nucleic acids. In certain embodiments, the vector is an expression vector. The presently disclosed subject matter further provides host cells comprising the expression vectors disclosed herein.

### 5.5. Pharmaceutical Compositions and Methods of Treatment

The presently disclosed subject matter provides compositions comprising a presently disclosed anti-CD371 antibody or an antigen-binding fragment thereof, a presently disclosed immunoconjugate, a presently disclosed bispecific antibody. In certain embodiments, the composition is a pharmaceutical composition further comprising a pharmaceutically acceptable carrier.

The presently disclosed subject matter provides various methods of using the anti-CD371 antibodies or antigen-binding fragments thereof, the immunoconjugate, the bispecific antibody, and the composition disclosed herein. For example, the presently disclosed subject matter provides methods of reducing tumor burden in a subject. In certain embodiments, the method comprises administering one or more of the anti-CD371 antibodies or antigen-binding fragments thereof, the immunoconjugate, the bispecific antibody, or the composition disclosed herein to the subject. The presently disclosed anti-CD371 antibodies or antigen-binding fragments thereof can reduce the number of tumor cells, reduce tumor size, and/or eradicate the tumor in the subject.

The presently disclosed subject matter also provides methods of increasing or lengthening survival of a subject having a tumor or neoplasm. In certain embodiments, the method comprises administering one or more of the anti-CD371 antibodies or antigen-binding fragments thereof, the immunoconjugate, the bispecific antibody, or the composition disclosed herein to the subject. The method can reduce or eradicate tumor burden in the subject.

The presently disclosed subject matter further provides methods for treating and/or preventing a tumor or neoplasm in a subject. In certain embodiments, the method comprises administering one or more of the anti-CD371 antibodies or antigen-binding fragments thereof, the immunoconjugate, the bispecific antibody, or the composition disclosed herein to the subject.

Such methods comprise administering the presently disclosed anti-CD371 antibodies or antigen-binding fragments thereof in an amount effective, a presently disclosed composition (e.g., a pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of neoplasms or tumors include acute myeloid leukemia (AML), multiple myeloma, Chronic Lymphocytic Leukemia (CLL), lymphoma (Hodgkin's lymphoma, non-Hodgkin's lymphoma), glioblastoma, myelodysplastic syndrome (MDS), and chronic myelogenous leukemia (CML), bone cancer, intestinal cancer, liver cancer, skin cancer, cancer of the head or neck, melanoma (cutaneous or intraocular malignant melanoma), renal cancer (e.g. clear cell carcinoma), throat cancer, prostate cancer (e.g. hormone refractory prostate adenocarcinoma), blood cancers (e.g. leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (e.g., acute leukemia, acute lymphocytic leukemia, acute myelocytic leukemia, acute myeloblastic leukemia, acute promyelocytic leukemia, acute monocytic leukemia, acute erythroleukemia, chronic leukemia, chronic myelocytic leukemia, polycythemia vera, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, include Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, nile duct carcinoma, choriocarcinoma, seminoma, embryonal carcinoma, Wilm's tumor, cervical cancer, salivary gland cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma.

Non-limiting examples of suitable tumors or neoplasms include acute myeloid leukemia (AML), multiple myeloma, Non-Hodgkin's Lymphoma, Hodgkin's Lymphoma, Chronic Lymphocytic Leukemia (CLL), glioblastoma, myelodysplastic syndrome (MDS), and chronic myelogenous leukemia (CML). In certain embodiments, the tumor or neoplasm is AML.

Any suitable method or route can be used to administer a presently disclosed anti-CD371 antibody, and optionally, to co-administer antineoplastic agents. Routes of administration include, but are not limited to, oral, intravenous, intraperitoneal, subcutaneous, intramuscular, intranodal, intratumoral, intraosseous, intrathecal, pleural, intrapleural, and direct administration. It should be emphasized, however, that the presently disclosed subject matter is not limited to any particular method or route of administration.

The presently disclosed anti-CD371 antibodies or antigen-binding fragments thereof can be administered as a conjugate, which binds specifically to the receptor and delivers a toxic, lethal payload following ligand-toxin internalization.

The anti-CD371 antibodies or antigen-binding fragments thereof of the presently disclosed subject matter can be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter also provides use of antibodies and nucleic acids that encode them for treatment of a tumor or neoplasm (e.g., AML), for diagnostic and prognostic applications as well as use as research tools for the detection of CD371 in cells and tissues. Pharmaceutical compositions comprising the disclosed antibodies and nucleic acids are encompassed by the presently disclosed subject matter. Vectors comprising the nucleic acids of the presently disclosed subject matter for antibody-based treatment by vectored immunotherapy are also contemplated by the presently disclosed subject matter. Vectors include expression vectors which enable the expression and secretion of antibodies, as well as vectors which are directed to cell surface expression of the antigen binding proteins, such as chimeric antigen receptors.

Cells comprising the nucleic acids, for example cells that have been transfected with the vectors of the invention are also encompassed by the presently disclosed subject matter.

### 5.6. Kits

The presently disclosed subject matter provides kits for the treatment and/or prevention of a tumor or neoplasm (e.g., AML), for reducing tumor burden, and/or for increasing or lengthening survival of a subject having a tumor or neoplasm (e.g., AML). In certain embodiments, the kit comprises a composition comprising the anti-CD371 antibodies or antigen-binding fragments thereof, the immunoconjugate, the bispecific antibody, or the composition disclosed herein in unit dosage form. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-CD371 antibodies or antigen-binding fragments thereof, the immunoconjugate, the bispecific antibody, or the composition disclosed herein to the subject. The instructions can generally include information about the use of the anti-CD371 antibodies or antigen-binding fragments thereof, the immunoconjugate, the bispecific antibody, and the composition disclosed herein for the treatment and/or prevention of a tumor or neoplasm (e.g., AML), for reducing tumor burden, and/or for increasing or lengthening survival of a subject having a tumor or neoplasm (e.g., AML). In certain embodiments, the instructions include at least one of the following: description of the therapeutic agent; dosage schedule and administration for treatment and/or prevention of a tumor or neoplasm (e.g., AML) or symptoms thereof, precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.7. Methods of Detection

The presently discloses subject matter provides methods for detecting CD371 in a whole cell or tissue. In certain embodiments, the method comprises:

a) contacting a cell or tissue with an anti-CD371 antibody or antigen-binding fragment disclosed herein, wherein the antibody or antigen-binding fragment thereof comprises a detectable label; and

b) determining the amount of the labeled antibody or antigen-binding fragment thereof bound to the cell or tissue.

In certain embodiments, b) comprises measuring the amount of detectable label associated with the cell or tissue, wherein the amount of bound antibody or antigen-binding fragment thereof indicates the amount of CD371 in the cell or tissue.

The cell or tissue can be any cell or tissue, including any normal, healthy, or cancerous cells and tissues.

### 6. EXAMPLES

The following examples are put forth so as to provide those of ordinary skill in the art with a complete disclosure and description of how to make and use the antibodies, bispecific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—Generation of Anti-CD371 Antibodies and scFvs

A portion of CD371 (UniProt accession number Q5QGZ9) corresponding to the extracellular domain and amino acids His 65-Ala 265, was recombinantly produced as a soluble protein with a polyhistidine tag for purification. The extracellular domain of murine CD371 (Thr 67-Arg 267) was also produced with a polyhistidine tag to screen antibodies for cross-species reactivity.

A proprietary naïve, semi-synthetic scFv phage display library was screened for antibodies that bind to the CD371 protein by using standard solid phase phage display panning techniques. Briefly, recombinant CD371 was immobilized on a polystyrene surface followed by blocking with about 5% milk and incubation with the phage library. Subsequent washing, elution and phage amplification steps were performed to complete each round of biopanning. Three rounds of panning were completed using amplified CD371 binder-enriched phage pools from the previous round of panning as input for subsequent rounds. In order to identify clones that showed high specificity for CD371, single clones from the third round of panning were analyzed for binding to human CD371, murine CD371, and BSA (as a non-specific control) by enzyme-linked immunosorbent assay (ELISA) using an anti-M13 phage antibody. Only those monoclonal phage supernatants that showed CD371-specific binding were selected for antibody sequencing, resulting in the identification of six antibodies with unique sequences (B10 (also referred to as “1B10”), C3 (also referred to as “1C3”), D6 (also referred to as “1D6”), A11 (also referred to as “2A11”), E4 (also referred to as “2E4”), and E8 (also referred to as “2E8”)). None of the antibodies screened showed binding to both human and mouse homologs of CD371.

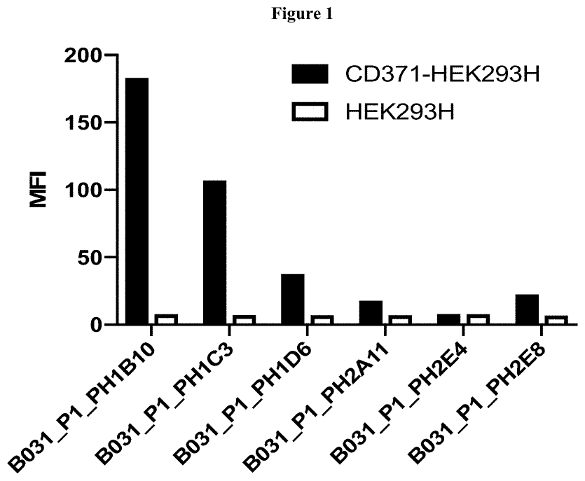

To test whether antibodies recovered from the phage panning campaign were able to bind to CD371 in its native conformation on the cell surface, monoclonal phage preps were also screened by flow cytometry on HEK293H cells transfected with CD371 and wild type HEK293H cells. FIG. 1 depicts the binding profile of the 1B10, 1C3, 1D6, 2A11, 2E4, and 2E8 antibodies.

### Example 2: Antibody Binding to CD371-Expressing Cell Lines

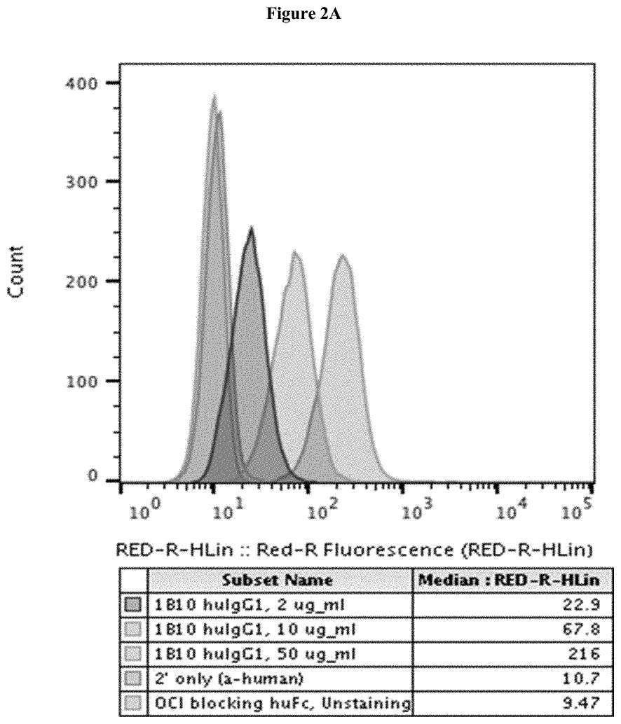

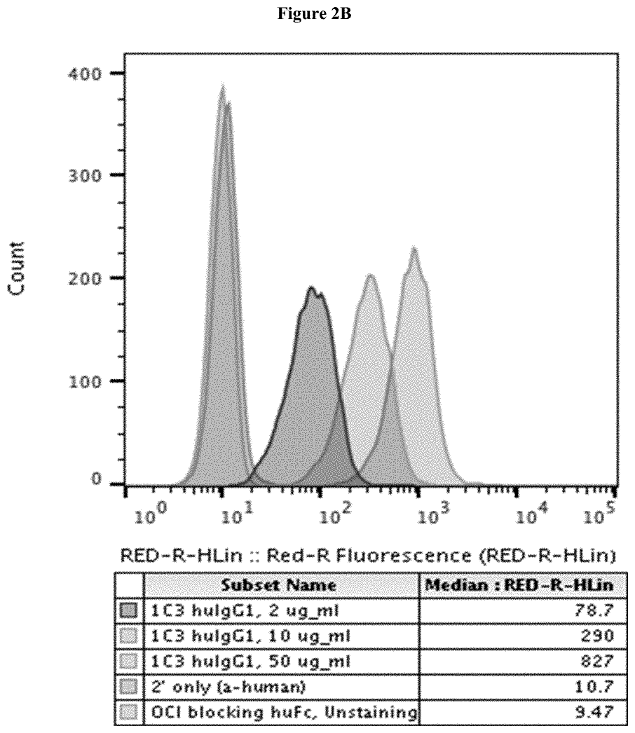

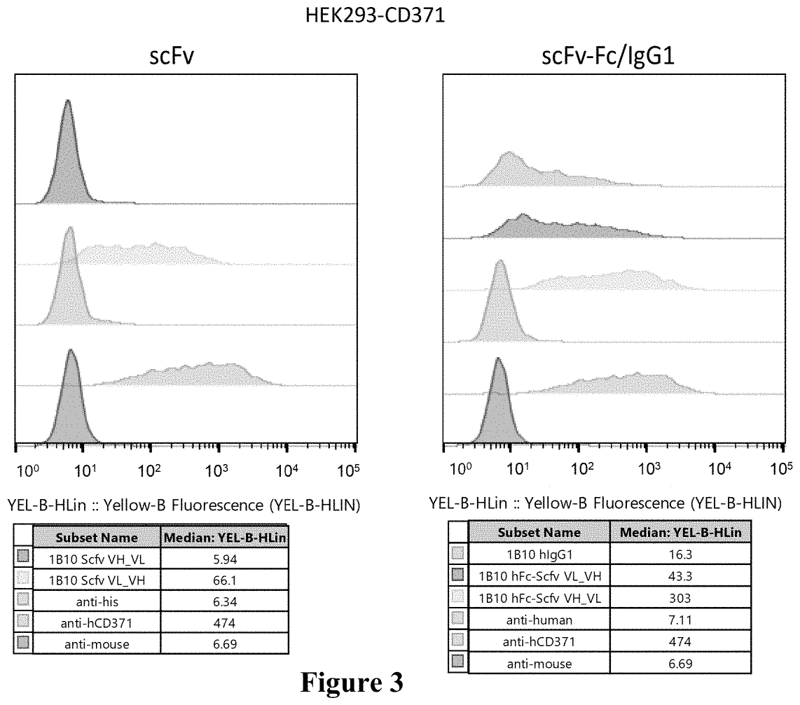

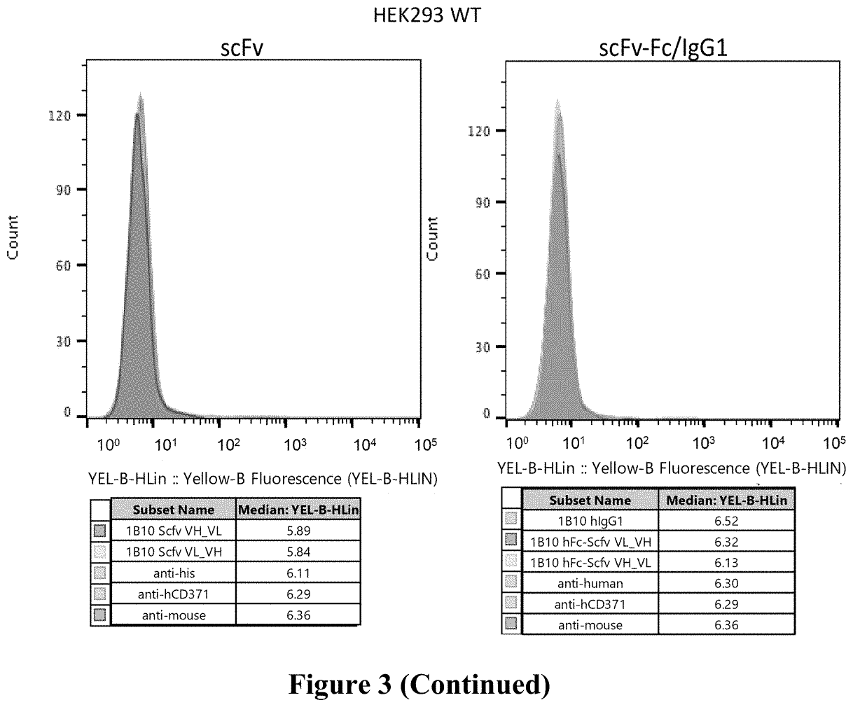

Based on preliminary in vitro functional characterization, two mAbs (1B10 and 1C3) were reformatted to human IgG1 and tested for binding to OCI cells, a CD371⁺ AML cell line. Both mAbs demonstrated dose-dependent binding as depicted in FIGS. 2A and 2B. B10 was further engineered into several formats including scFv-Fc fusions with the variable domains in both orientations (V_{H}-V_{L} or V_{L}-V_{H}). These scFv-Fc fusion constructs showed specific binding to CD371 cells (see FIG. 3). Similarly, binding to cells was detected for the B10 scFv in the V_{L}-V_{H} orientation. However, binding of the B10 scFv in the V_{H}-V_{L} orientation was not observed by flow cytometry, suggesting a lower affinity and thus a requirement for bivalent binding.

### Example 3: Antibody Binding to Recombinant CD371 in Solution

Affinity measurements of the B10 variants determined by biolayer interferometry by capturing the IgG and Fc fusions with anti-Fc antibody, and using soluble CD371 as analyte. For scFv affinity measurements, biotinylated CD371 was captured with streptavidin, and soluble scFv was used as analyte. Table 8 shows dissociation constants (K_{D}), on-rates (kₒₙ) and off-rates (k_{off}) for the different antibody formats. Consistent with the flow cytometry results, the full IgG and the scFv-Fc fusions bound more strongly than the scFvs (Table 8), presumably due to their bivalent interaction, resulting in an avidity effect. Weak binding of the 1B10 scFv in the V_{H}-V_{L} Orientation was observed but a dissociation constant could not be calculated by any curve fit method.

TABLE 8
Binding affinity of Antibody B10 in various formats to soluble CD371

| Format | KD (nM) | Kₒₙ (1/MS) | K_{off} (1/s) |
|---|---|---|---|
| IgG1 | 9.5 | 1.60 × 10⁵ | 1.53 × 10⁻³ |
| V_{L}-V_{H}-Fc | 7.0 | 2.95 × 10⁵ | 2.06 × 10⁻³ |
| V_{H}-V_{L}-Fc | 4.2 | 2.32 × 10⁵ | 9.68 × 10⁻⁴ |
| V_{L}-V_{H} scFv | 16 | 5.99 × 10⁵ | 9.52 × 10⁻³ |
| V_{H}-V_{L} scFv | Weak binding | NA | NA |

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An anti-CD371 antibody or an antigen-binding fragment thereof, comprising a heavy chain variable region and a light chain variable region, wherein:
(a) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 1, SEQ ID NO: 3, SEQ ID NO: 5, SEQ ID NO: 7, SEQ ID NO: 9, or SEQ ID NO: 11; and/or
(b) the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 2, SEQ ID NO: 4, SEQ ID NO: 6, SEQ ID NO: 8, SEQ ID NO: 10, or SEQ ID NO: 12.

2. The antibody or the antigen-binding fragment thereof of claim 1, wherein:
(a) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:1, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:2;
(b) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 3, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 4;
(c) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 5, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO:6;
(d) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 7, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 8;
(e) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 9, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 10; or
(f) the heavy chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 11, and the light chain variable region comprises an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 12.

3. The antibody or the antigen-binding fragment thereof of claim 1, wherein:
(a) the heavy chain variable region comprises the amino acid sequence set forth in SEQ ID NO: 1, SEQ ID NO: 3, SEQ ID NO: 5, SEQ ID NO: 7, SEQ ID NO: 9, or SEQ ID NO: 11; and/or
(b) the light chain variable region comprises the amino acid sequence set forth in SEQ ID NO: 2, SEQ ID NO: 4, SEQ ID NO: 6, SEQ ID NO: 8, SEQ ID NO: 10, or SEQ ID NO: 12.

4. The antibody or the antigen-binding fragment thereof of claim 1, wherein:
(a) the heavy chain variable region comprises the amino acid sequence set forth in SEQ ID NO: 1, and the light chain variable region comprises the amino acid sequence set forth in SEQ ID NO: 2;
(b) the heavy chain variable region comprises the amino acid sequence set forth in SEQ ID NO: 3, and the light chain variable region comprises the amino acid sequence set forth in SEQ ID NO: 4;
(c) the heavy chain variable region comprises the amino acid sequence set forth in SEQ ID NO: 5, and the light chain variable region comprises the amino acid sequence set forth in SEQ ID NO: 6;
(d) the heavy chain variable region comprises the amino acid sequence set forth in SEQ ID NO: 7, and the light chain variable region comprises the amino acid sequence set forth in SEQ ID NO: 8;
(e) the heavy chain variable region comprises the amino acid sequence set forth in SEQ ID NO:9, and the light chain variable region comprises the amino acid sequence set forth in SEQ ID NO: 10; or
(f) the heavy chain variable region comprises the amino acid sequence set forth in SEQ ID NO: 11, and the light chain variable region comprises the amino acid sequence set forth in SEQ ID NO: 12.

5. An anti-CD371 antibody or an antigen-binding fragment thereof, comprising a heavy chain variable region that comprises CDR1, CDR2, and CDR3; and a light chain variable region that comprises CDR1, CDR2, and CDR3 domains, wherein:
(a) the heavy chain variable region CDR3 comprises the amino acid sequence set forth in SEQ ID NO: 30 or a conservative modification thereof, and the light chain variable region CDR3 comprises the amino acid sequence set forth in SEQ ID NO: 33 or a conservative modification thereof,
(b) the heavy chain variable region CDR3 comprises the amino acid sequence set forth in SEQ ID NO: 36 or a conservative modification thereof, and the light chain variable region CDR3 comprises the amino acid sequence set forth in SEQ ID NO: 39 or a conservative modification thereof,
(c) the heavy chain variable region CDR3 comprises the amino acid sequence set forth in SEQ ID NO: 42 or a conservative modification thereof, and the light chain variable region CDR3 comprises the amino acid sequence set forth in SEQ ID NO: 45 or a conservative modification thereof,
(d) the heavy chain variable region CDR3 comprises the amino acid sequence set forth in SEQ ID NO: 48 or a conservative modification thereof, and the light chain variable region CDR3 comprises the amino acid sequence set forth in SEQ ID NO: 51 or a conservative modification thereof,
(e) the heavy chain variable region CDR3 comprises the amino acid sequence set forth in SEQ ID NO: 54 or a conservative modification thereof, and the light chain variable region CDR3 comprises the amino acid sequence set forth in SEQ ID NO: 57 or a conservative modification thereof, or
(f) the heavy chain variable region CDR3 comprises the amino acid sequence set forth in SEQ ID NO: 60 or a conservative modification thereof, and the light chain variable region CDR3 comprises the amino acid sequence set forth in SEQ ID NO: 63 or a conservative modification thereof.

6. The antibody or the antigen-binding fragment thereof of claim 5, wherein:
(a) the heavy chain variable region CDR2 comprises the amino acid sequence set forth in SEQ ID NO: 29 or a conservative modification thereof, and the light chain variable region CDR2 comprises the amino acid sequence set forth in SEQ ID NO: 32 or a conservative modification thereof,
(b) the heavy chain variable region CDR2 comprises the amino acid sequence set forth in SEQ ID NO: 35 or a conservative modification thereof, and the light chain variable region CDR2 comprises the amino acid sequence set forth in SEQ ID NO: 38 or a conservative modification thereof,
(c) the heavy chain variable region CDR2 comprises the amino acid sequence set forth in SEQ ID NO: 41 or a conservative modification thereof, and the light chain variable region CDR2 comprises the amino acid sequence set forth in SEQ ID NO: 44 or a conservative modification thereof,
(d) the heavy chain variable region CDR2 comprises the amino acid sequence set forth in SEQ ID NO: 47 or a conservative modification thereof, and the light chain variable region CDR2 comprises the amino acid sequence set forth in SEQ ID NO: 50 or a conservative modification thereof,
(e) the heavy chain variable region CDR2 comprises the amino acid sequence set forth in SEQ ID NO: 53 or a conservative modification thereof, and the light chain variable region CDR2 comprises the amino acid sequence set forth in SEQ ID NO: 56 or a conservative modification thereof, or
(f) the heavy chain variable region CDR2 comprises the amino acid sequence set forth in SEQ ID NO: 59 or a conservative modification thereof, and the light chain variable region CDR2 comprises the amino acid sequence set forth in SEQ ID NO: 62 or a conservative modification thereof.

7. The antibody or the antigen-binding fragment thereof of claim 5, wherein the heavy chain variable region and light chain variable region CDR1 domains are selected from:
(a) the heavy chain variable region CDR1 comprises the amino acid sequence set forth in SEQ ID NO: 28 or a conservative modification thereof, and the light chain variable region CDR1 comprises the amino acid sequence set forth in SEQ ID NO: 31 or a conservative modification thereof,
(b) the heavy chain variable region CDR1 comprises the amino acid sequence set forth in SEQ ID NO: 34 or a conservative modification thereof, and the light chain variable region CDR1 comprises the amino acid sequence set forth in SEQ ID NO: 37 or a conservative modification thereof,
(c) the heavy chain variable region CDR1 comprises the amino acid sequence set forth in SEQ ID NO: 40 or a conservative modification thereof, and the light chain variable region CDR1 comprises the amino acid sequence set forth in SEQ ID NO: 43 or a conservative modification thereof,
(d) the heavy chain variable region CDR1 comprises the amino acid sequence set forth in SEQ ID NO: 46 or a conservative modification thereof, and the light chain variable region CDR1 comprises the amino acid sequence set forth in SEQ ID NO: 49 or a conservative modification thereof,
(e) the heavy chain variable region CDR1 comprises the amino acid sequence set forth in SEQ ID NO: 52 or a conservative modification thereof, and the light chain variable region CDR1 comprises the amino acid sequence set forth in SEQ ID NO: 55 or a conservative modification thereof, or
(f) the heavy chain variable region CDR1 comprises the amino acid sequence set forth in SEQ ID NO: 58 or a conservative modification thereof, and the light chain variable region CDR1 comprises the amino acid sequence set forth in SEQ ID NO: 61 or a conservative modification thereof.

8. The antibody or the antigen-binding fragment thereof of claim 5, wherein one or more of the CDR sequences have up to about 5 or about 3 amino acid substitutions.

9. The anti-CD371 antibody or the antigen-binding fragment thereof of claim 5, wherein:
(a) the heavy chain variable region CDR1 comprises the amino acid sequence set forth in SEQ ID NO: 28; the heavy chain variable region CDR2 comprises the amino acid sequence set forth in SEQ ID NO: 29; and the heavy chain variable region CDR3 comprises the amino acid sequence set forth in SEQ ID NO: 30;
(b) the heavy chain variable region CDR1 comprises the amino acid sequence set forth in SEQ ID NO: 34; the heavy chain variable region CDR2 comprises the amino acid sequence set forth in SEQ ID NO: 35; and the heavy chain variable region CDR3 comprises the amino acid sequence set forth in SEQ ID NO: 36;
(c) the heavy chain variable region CDR1 comprises the amino acid sequence set forth in SEQ ID NO: 40; the heavy chain variable region CDR2 comprises the amino acid sequence set forth in SEQ ID NO: 41; and the heavy chain variable region CDR3 comprises the amino acid sequence set forth in SEQ ID NO: 42;
(d) the heavy chain variable region CDR1 comprises the amino acid sequence set forth in SEQ ID NO: 46; the heavy chain variable region CDR2 comprises the amino acid sequence set forth in SEQ ID NO: 47; and the heavy chain variable region CDR3 comprises the amino acid sequence set forth in SEQ ID NO: 48;
(e) the heavy chain variable region CDR1 comprises the amino acid sequence set forth in SEQ ID NO: 52; the heavy chain variable region CDR2 comprises the amino acid sequence set forth in SEQ ID NO: 53; and the heavy chain variable region CDR3 comprises the amino acid sequence set forth in SEQ ID NO: 54, or
(f) the heavy chain variable region CDR1 comprises the amino acid sequence set forth in SEQ ID NO: 58; the heavy chain variable region CDR2 comprises the amino acid sequence set forth in SEQ ID NO: 59; and the heavy chain variable region CDR3 comprises the amino acid sequence set forth in SEQ ID NO: 60.

10. The antibody or the antigen-binding fragment thereof of claim 5, wherein:
(a) the light chain variable region CDR1 comprises the amino acid sequence set forth in SEQ ID NO: 31; the light chain variable region CDR2 comprises the amino acid sequence set forth in SEQ ID NO: 32; and the light chain variable region CDR3 comprises the amino acid sequence set forth in SEQ ID NO: 33;
(b) the light chain variable region CDR1 comprises the amino acid sequence set forth in SEQ ID NO: 37; the light chain variable region CDR2 comprises the amino acid sequence set forth in SEQ ID NO: 38; and the light chain variable region CDR3 comprises the amino acid sequence set forth in SEQ ID NO: 38;
(c) the light chain variable region CDR1 comprises the amino acid sequence set forth in SEQ ID NO: 43; the light chain variable region CDR2 comprises the amino acid sequence set forth in SEQ ID NO: 44; and the light chain variable region CDR3 comprises the amino acid sequence set forth in SEQ ID NO: 45;
(d) the light chain variable region CDR1 comprises the amino acid sequence set forth in SEQ ID NO: 49; the light chain variable region CDR2 comprises the amino acid sequence set forth in SEQ ID NO: 50; and the light chain variable region CDR3 comprises the amino acid sequence set forth in SEQ ID NO: 51;
(e) the light chain variable region CDR1 comprises the amino acid sequence set forth in SEQ ID NO: 55; the light chain variable region CDR2 comprises the amino acid sequence set forth in SEQ ID NO: 56; and the light chain variable region CDR3 comprises the amino acid sequence set forth in SEQ ID NO: 57, or
(f) the light chain variable region CDR1 comprises the amino acid sequence set forth in SEQ ID NO: 61; the light chain variable region CDR2 comprises the amino acid sequence set forth in SEQ ID NO: 62; and the light chain variable region CDR3 comprises the amino acid sequence set forth in SEQ ID NO: 63.

11. The antibody or the antigen-binding fragment thereof of claim 5, wherein:
(a) the heavy chain variable region CDR1 comprises the amino acid sequence set forth in SEQ ID NO: 28; the heavy chain variable region CDR2 comprises the amino acid sequence set forth in SEQ ID NO: 29; the heavy chain variable region CDR3 comprises the amino acid sequence set forth in SEQ ID NO: 30; the light chain variable region CDR1 comprises the amino acid sequence set forth in SEQ ID NO: 31; the light chain variable region CDR2 comprises the amino acid sequence set forth in SEQ ID NO: 32; and the light chain variable region CDR3 comprises the amino acid sequence set forth in SEQ ID NO: 33;
(b) the heavy chain variable region CDR1 comprises the amino acid sequence set forth in SEQ ID NO: 34; the heavy chain variable region CDR2 comprises an amino acid sequence set forth in SEQ ID NO: 35; the heavy chain variable region CDR3 comprises the amino acid sequence set forth in SEQ ID NO: 36; the light chain variable region CDR1 comprises the amino acid sequence set forth in SEQ ID NO: 37; the light chain variable region CDR2 comprises the amino acid sequence set forth in SEQ ID NO: 38; and the light chain variable region CDR3 comprises the amino acid sequence set forth in SEQ ID NO: 39;
(c) the heavy chain variable region CDR1 comprises the amino acid sequence set forth in SEQ ID NO: 40; the heavy chain variable region CDR2 comprises the amino acid sequence set forth in SEQ ID NO: 41; the heavy chain variable region CDR3 comprises the amino acid sequence set forth in SEQ ID NO: 42; the light chain variable region CDR1 comprises the amino acid sequence set forth in SEQ ID NO: 43; the light chain variable region CDR2 comprises the amino acid sequence set forth in SEQ ID NO: 44; and the light chain variable region CDR3 comprises the amino acid sequence set forth in SEQ ID NO: 45;
(d) the heavy chain variable region CDR1 comprises the amino acid sequence set forth in SEQ ID NO: 46; the heavy chain variable region CDR2 comprises the amino acid sequence set forth in SEQ ID NO: 47; the heavy chain variable region CDR3 comprises the amino acid sequence set forth in SEQ ID NO: 48; the light chain variable region CDR1 comprises the amino acid sequence set forth in SEQ ID NO: 49; the light chain variable region CDR2 comprises the amino acid sequence set forth in SEQ ID NO: 50; and the light chain variable region CDR3 comprises the amino acid sequence set forth in SEQ ID NO: 51;
(e) the heavy chain variable region CDR1 comprises the amino acid sequence set forth in SEQ ID NO: 52; the heavy chain variable region CDR2 comprises the amino acid sequence set forth in SEQ ID NO: 53; the heavy chain variable region CDR3 comprises the amino acid sequence set forth in SEQ ID NO: 54; the light chain variable region CDR1 comprises the amino acid sequence set forth in SEQ ID NO: 55; the light chain variable region CDR2 comprises the amino acid sequence set forth in SEQ ID NO: 56; and the light chain variable region CDR3 comprises the amino acid sequence set forth in SEQ ID NO: 57; or
(f) the heavy chain variable region CDR1 comprises the amino acid sequence set forth in SEQ ID NO: 58; the heavy chain variable region CDR2 comprises the amino acid sequence set forth in SEQ ID NO: 59; the heavy chain variable region CDR3 comprises the amino acid sequence set forth in SEQ ID NO: 60; the light chain variable region CDR1 comprises the amino acid sequence set forth in SEQ ID NO: 61; the light chain variable region CDR2 comprises the amino acid sequence set forth in SEQ ID NO: 62; and the light chain variable region CDR3 comprises the amino acid sequence set forth in SEQ ID NO: 63.

12. The antibody or the antigen-binding fragment thereof of claim 1, wherein the antibody or antigen-binding fragment thereof binds to a CD371 comprising the amino acid sequence set forth in SEQ ID NO: 15 or a fragment thereof.

13. An antibody or an antigen-binding fragment thereof, which (i) cross-competes for binding to CD371 with the antibody or the antigen-binding fragment thereof of claim 1; or (ii) binds to the same epitope on CD371 as the antibody or the antigen-binding fragment thereof of claim 1.

14. The antibody or the antigen-binding fragment thereof of claim 1, wherein the sequence of the antibody is in a light-heavy variable chain orientation (V_{L}-V_{H}).

15. The antibody or the antigen-binding fragment thereof of claim 1, wherein the antibody or the antigen-binding fragment thereof binds to human CD371 with a dissociation constant (K_{D}) of between about 1×10⁻⁷ M and about 1×10⁻⁸ M or between about 1×10⁻⁹ M and about 1×10⁻⁸ M.

16. The antibody or the antigen-binding fragment thereof of claim 1, wherein the antibody or the antigen-binding fragment thereof comprises the amino acid sequence set forth in SEQ ID NO: 16, SEQ ID NO: 17, SEQ ID NO: 18, SEQ ID NO: 19, SEQ ID NO: 20, or SEQ ID NO: 21.

17. The antibody or the antigen-binding fragment thereof of claim 1, wherein the antibody comprises a human variable region framework region.

18. The antibody or the antigen-binding fragment thereof of claim 1, which is a fully human antibody or an antigen-binding fragment thereof, a chimeric antibody or an antigen-binding fragment thereof, or a humanized antibody or an antigen-binding fragment thereof.

19. The antibody or the antigen-binding fragment thereof of claim 1, wherein the antigen-binding fragment of the antibody is a Fab, Fab′, F(ab′)₂, a variable fragment (Fv), or a single chain variable region (scFv).

20. A composition comprising the antibody or the antigen-binding fragment thereof of claim 1.

21. An immunoconjugate comprising the antibody or the antigen-binding fragment thereof of claim 1, linked to a therapeutic agent.

22. A composition comprising the immunoconjugate of claim 21.

23. A bispecific molecule comprising the antibody or the antigen-binding fragment thereof of claim 1, linked to a second functional moiety.

24. A composition comprising the bispecific molecule of claim 23.

25. A nucleic acid that encodes the antibody or the antigen-binding fragment thereof of claim 1.

26. An expression vector comprising the nucleic acid of claim 25.

27. A host cell comprising the expression vector of claim 26.

28. A method for detecting CD371 in a whole cell or tissue, comprising:
contacting a cell or tissue with the antibody or the antigen-binding fragment thereof of claim 1, wherein the antibody or the antigen-binding fragment thereof comprises a detectable label; and
determining the amount of the labeled antibody or the antigen-binding fragment thereof bound to said cell or tissue by measuring the amount of detectable label associated with said cell or tissue, wherein the amount of bound antibody or the antigen-binding fragment thereof indicates the amount of CD371 in said cell or tissue.

29. A method of reducing tumor burden in a subject, comprising administering to the subject the antibody or the antigen-binding fragment thereof of claim 1.

30. A method of treating and/or preventing a tumor or neoplasm in a subject, comprising administering to the subject the antibody or the antigen-binding fragment thereof of claim 1.

31. A method of increasing or lengthening survival of a subject having a tumor or neoplasm, comprising administering to the subject the antibody or the antigen-binding fragment thereof of claim 1.

32. A kit for reducing tumor burden in a subject, treating and/or preventing a tumor or neoplasm in a subject, and/or increasing or lengthening survival of a subject having a tumor or neoplasm, comprising the antibody or the antigen-binding fragment thereof of claim 1.


SEQUENCE LISTING (73 sequences)

SEQ ID NO: 1 - protein, 122 aa, Artificial Sequence
  Synthetic polypeptide
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 2 - protein, 113 aa, Artificial Sequence
  Synthetic polypeptide
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 3 - protein, 125 aa, Artificial Sequence
  Synthetic polypeptide
EVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSS

SEQ ID NO: 4 - protein, 113 aa, Artificial Sequence
  Synthetic polypeptide
DIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIK

SEQ ID NO: 5 - protein, 117 aa, Artificial Sequence
  Synthetic polypeptide
EVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSS

SEQ ID NO: 6 - protein, 113 aa, Artificial Sequence
  Synthetic polypeptide
DIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIK

SEQ ID NO: 7 - protein, 120 aa, Artificial Sequence
  Synthetic polypeptide
EVQLLESGGGLVQPGGSLRLSCAASGFTFTSTQMSWVRQAPGKGLEWVSEISGYGGSTYYADSVKGRFTISRDNSKNTLYLQMNSLRAEDTAVYYCAKDTEVSGDAFDIWGQGTMVTVSS

SEQ ID NO: 8 - protein, 108 aa, Artificial Sequence
  Synthetic polypeptide
EIVLTQSPGTLSLSPGERATLSCRASQSVDSSNLAWYQQKPGQAPRLLIYGASSRATGIPDRFSGSGSGTDFTLTISRLEPEDFAVYYCQQYRSWPITFGQGTKVEIK

SEQ ID NO: 9 - protein, 122 aa, Artificial Sequence
  Synthetic polypeptide
EVQLLESGGGLVQPGGSLRLSCAASGFTFTSYYMSWVRQAPGKGLEWVSGISGSGDSTSYADSVKGRFTISRDNSKNTLYLQMNSLRAEDTAVYYCAREAGGDYDSGAFDIWGQGTMVTVSS

SEQ ID NO: 10 - protein, 113 aa, Artificial Sequence
  Synthetic polypeptide
DIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYDYAPFTFGQGTKVEIK

SEQ ID NO: 11 - protein, 127 aa, Artificial Sequence
  Synthetic polypeptide
EVQLLESGGGLVQPGGSLRLSCAASGFTFSSYAMSWVRQAPGKGLEWVSEIDGEGGYTNYADSVKGRFTISRDNSKNTLYLQMNSLRAEDTAVYYCAREGVDYDILTGYYPYGMDVWGQGTTVTVSS

SEQ ID NO: 12 - protein, 114 aa, Artificial Sequence
  Synthetic polypeptide
DIVMTQSPDSLAVSLGERATINCKSSQSVLDSSNNKNYLAWYQQKPGQPPKLLIYDASTRESGVPDRFSGSGSGTDFTLTISSLQAEDVAVYYCQQGTSSPLTFGQGTKVEIKG

SEQ ID NO: 13 - protein, 19 aa, Artificial Sequence
  Synthetic polypeptide
GGGGSGGGGSGGGSGGGGS

SEQ ID NO: 14 - protein, 15 aa, Artificial Sequence
  Synthetic polypeptide
GGGGSGGGGSGGGGS

SEQ ID NO: 15 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 16 - protein, 254 aa, Artificial Sequence
  Synthetic polypeptide
DIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIKGGGGSGGGGSGGGSGGGGSEVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSS

SEQ ID NO: 17 - protein, 257 aa, Artificial Sequence
  Synthetic polypeptide
DIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIKGGGGSGGGGSGGGSGGGGSEVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSS

SEQ ID NO: 18 - protein, 249 aa, Artificial Sequence
  Synthetic polypeptide
DIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIKGGGGSGGGGSGGGSGGGGSEVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSS

SEQ ID NO: 19 - protein, 247 aa, Artificial Sequence
  Synthetic polypeptide
EIVLTQSPGTLSLSPGERATLSCRASQSVDSSNLAWYQQKPGQAPRLLIYGASSRATGIPDRFSGSGSGTDFTLTISRLEPEDFAVYYCQQYRSWPITFGQGTKVEIKGGGGSGGGGSGGGSGGGGSEVQLLESGGGLVQPGGSLRLSCAASGFTFTSTQMSWVRQAPGKGLEWVSEISGYGGSTYYADSVKGRFTISRDNSKNTLYLQMNSLRAEDTAVYYCAKDTEVSGDAFDIWGQGTMVTVSS

SEQ ID NO: 20 - protein, 254 aa, Artificial Sequence
  Synthetic polypeptide
DIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYDYAPFTFGQGTKVEIKGGGGSGGGGSGGGSGGGGSEVQLLESGGGLVQPGGSLRLSCAASGFTFTSYYMSWVRQAPGKGLEWVSGISGSGDSTSYADSVKGRFTISRDNSKNTLYLQMNSLRAEDTAVYYCAREAGGDYDSGAFDIWGQGTMVTVSS

SEQ ID NO: 21 - protein, 259 aa, Artificial Sequence
  Synthetic polypeptide
DIVMTQSPDSLAVSLGERATINCKSSQSVLDSSNNKNYLAWYQQKPGQPPKLLIYDASTRESGVPDRFSGSGSGTDFTLTISSLQAEDVAVYYCQQGTSSPLTFGQGTKVEIKGGGGSGGGGSGGGSGGGGSEVQLLESGGGLVQPGGSLRLSCAASGFTFSSYAMSWVRQAPGKGLEWVSEIDGEGGYTNYADSVKGRFTISRDNSKNTLYLQMNSLRAEDTAVYYCAREGVDYDILTGYYPYGMDVWGQGTTVTVSS

SEQ ID NO: 22 - DNA, 762 bp, Artificial Sequence
  Synthetic polynucleotide
gacatcgtgatgacccagtctccagactccctggctgtgtctctgggcgagcgtgccaccatcaactgcaagtccagccagagtgttttagacagctataacaatgagaacaatttagcttggtatcagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatataccagcgaacctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcgactatcagatgagctgggtccgccaggctccagggaaggggctggagtgggtgtcaggcattcagggtggcggtggtagcacatattacgcagactccgtgaagggccggttcaccatctcccgtgacaattccaagaacacgctgtatctgcaaatgaacagcctgcgtgccgaggacacggctgtgtattactgtgcgagagagatgtggcgtggggactactactccggtatggacgtctggggccaggggaccacggtcaccgtctcctca

SEQ ID NO: 23 - DNA, 771 bp, Artificial Sequence
  Synthetic polynucleotide
gacatcgtgatgacccagtctccagactccctggctgtgtctctgggcgagcgtgccaccatcaactgcaagtccagccagagtgttttaagcagctataacaatgagaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatagcgaaccttatacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccagctatgccatgagctgggtccgccaggctccagggaaggggctggagtgggtgtcaggcattgacggtagcggtggtggcacaaattacgcagactccgtgaagggccggttcaccatctcccgtgacaattccaagaacacgctgtatctgcaaatgaacagcctgcgtgccgaggacacggctgtgtattactgtgcgagagcgtattacgatattttgactggttaccccgtggacggtatggacgtctggggccaagggaccacggtcaccgtctcctca

SEQ ID NO: 24 - DNA, 747 bp, Artificial Sequence
  Synthetic polynucleotide
gacatcgtgatgacccagtctccagactccctggctgtgtctctgggcgagcgtgccaccatcaactgcaagtccagccagagtgttttacgcagcagcaacaataaaaacaatttagcttggtatcagcagaaaccaggacagcctcctaagctgctcatttacgccgcatctacccgggaatccggggtccctgaccgattcagtggcagcgggtctgggacagatttcactctcaccatcagcagcctgcaggctgaagatgtggcagtttattactgtcagcaatattatcgcgaacctctgacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccgactatgccatgagctgggtccgccaggctccagggaaggggctggagtgggtgtcagacattgacggtagcggtggtagcacagactacgcagactccgtgaagggccggttcaccatctcccgtgacaattccaagaacacgctgtatctgcaaatgaacagcctgcgtgccgaggacacggctgtgtattactgtgcgctagagctgggagctactaccgtctactggggccagggaaccctggtcaccgtctcctca

SEQ ID NO: 25 - DNA, 741 bp, Artificial Sequence
  Synthetic polynucleotide
gaaattgtgttgacgcagtctccaggcaccctgtctttgtctccaggggaacgtgccaccctctcctgccgtgccagtcagagtgttgacagcagcaatttagcctggtatcagcagaaacctggccaggctccccgactcctcatctatggcgcatctagccgtgccactggtatcccagaccgtttcagtggcagtgggtctgggacagacttcactctcaccatcagcagactggagcctgaagattttgcagtgtattactgtcagcagtatcgcagctggcctatcacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccagcacccagatgagctgggtccgccaggctccagggaaggggctggagtgggtgtcagagattagcggttatggtggtagcacatactacgcagactccgtgaagggccggttcaccatctcccgtgacaattccaagaacacgctgtatctgcaaatgaacagcctgcgtgccgaggacacggctgtgtattactgtgcaaaagacacggaggtttcgggagatgcttttgatatctggggccaagggacaatggtcaccgtctcttca

SEQ ID NO: 26 - DNA, 762 bp, Artificial Sequence
  Synthetic polynucleotide
gacatcgtgatgacccagtctccagactccctggctgtgtctctgggcgagcgtgccaccatcaactgcaagtccagccagagtgttttatatagcggcaacaataaaaactatttagcttggtatcagcagaaaccaggacagcctcctaagctgctcatttacggcgcatctacccgggaatccggggtccctgaccgattcagtggcagcgggtctgggacagatttcactctcaccatcagcagcctgcaggctgaagatgtggcagtttattactgtcagcaatatgactatgccccttttacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttaccagctattatatgagctgggtccgccaggctccagggaaggggctggagtgggtgtcaggcattagcggtagcggtgacagcacaagctacgcagactccgtgaagggccggttcaccatctcccgtgacaattccaagaacacgctgtatctgcaaatgaacagcctgcgtgccgaggacacggctgtgtattactgtgcgagagaggcaggtggtgactacgatagtggtgcttttgatatctggggccaagggacaatggtcaccgtctcttca

SEQ ID NO: 27 - DNA, 777 bp, Artificial Sequence
  Synthetic polynucleotide
gacatcgtgatgacccagtctccagactccctggctgtgtctctgggcgagcgtgccaccatcaactgcaagtccagccagagtgttttagacagcagcaacaataaaaactatttagcttggtatcagcagaaaccaggacagcctcctaagctgctcatttacgacgcatctacccgggaatccggggtccctgaccgattcagtggcagcgggtctgggacagatttcactctcaccatcagcagcctgcaggctgaagatgtggcagtttattactgtcagcaaggcaccagcagccctctgacgttcggccaaggtaccaaggtggaaatcaaaggtggtggtggttcaggtggtggtggttctggcggcggctccggtggtggtggatccgaggtgcagctgttggagtctgggggaggcttggtacagcctggggggtccctgcgactctcctgtgcagcctctggattcacctttagcagctatgccatgagctgggtccgccaggctccagggaaggggctggagtgggtgtcagagattgacggtgagggtggttatacaaattacgcagactccgtgaagggccggttcaccatctcccgtgacaattccaagaacacgctgtatctgcaaatgaacagcctgcgtgccgaggacacggccgtgtattactgtgcgagagaaggggtagattacgatattttgactggttattatccttacggtatggacgtctggggccaagggaccacggtcaccgtctcctca

SEQ ID NO: 28 - protein, 8 aa, Artificial Sequence
  Synthetic polypeptide
GFTFSDYQ

SEQ ID NO: 29 - protein, 8 aa, Artificial Sequence
  Synthetic polypeptide
IQGGGGST

SEQ ID NO: 30 - protein, 15 aa, Artificial Sequence
  Synthetic polypeptide
AREMWRGDYYSGMDV

SEQ ID NO: 31 - protein, 12 aa, Artificial Sequence
  Synthetic polypeptide
QSVLDSYNNENN

SEQ ID NO: 32 - protein, 3 aa, Artificial Sequence
  Synthetic polypeptide
WAS

SEQ ID NO: 33 - protein, 9 aa, Artificial Sequence
  Synthetic polypeptide
QQYTSEPIT

SEQ ID NO: 34 - protein, 8 aa, Artificial Sequence
  Synthetic polypeptide
GFTFTSYA

SEQ ID NO: 35 - protein, 8 aa, Artificial Sequence
  Synthetic polypeptide
IDGSGGGT

SEQ ID NO: 36 - protein, 8 aa, Artificial Sequence
  Synthetic polypeptide
ARAYYDIL

SEQ ID NO: 37 - protein, 12 aa, Artificial Sequence
  Synthetic polypeptide
QSVLSSYNNENN

SEQ ID NO: 38 - protein, 3 aa, Artificial Sequence
  Synthetic polypeptide
AAS

SEQ ID NO: 39 - protein, 9 aa, Artificial Sequence
  Synthetic polypeptide
QQYYSEPYT

SEQ ID NO: 40 - protein, 8 aa, Artificial Sequence
  Synthetic polypeptide
GFTFTDYA

SEQ ID NO: 41 - protein, 8 aa, Artificial Sequence
  Synthetic polypeptide
IDGSGGST

SEQ ID NO: 42 - protein, 10 aa, Artificial Sequence
  Synthetic polypeptide
ALELGATTVY

SEQ ID NO: 43 - protein, 12 aa, Artificial Sequence
  Synthetic polypeptide
QSVLRSSNNKNN

SEQ ID NO: 44 - protein, 3 aa, Artificial Sequence
  Synthetic polypeptide
AAS

SEQ ID NO: 45 - protein, 9 aa, Artificial Sequence
  Synthetic polypeptide
QQYYREPLT

SEQ ID NO: 46 - protein, 8 aa, Artificial Sequence
  Synthetic polypeptide
GFTFTSTQ

SEQ ID NO: 47 - protein, 8 aa, Artificial Sequence
  Synthetic polypeptide
ISGYGGST

SEQ ID NO: 48 - protein, 13 aa, Artificial Sequence
  Synthetic polypeptide
AKDTEVSGDAFDI

SEQ ID NO: 49 - protein, 7 aa, Artificial Sequence
  Synthetic polypeptide
QSVDSSN

SEQ ID NO: 50 - protein, 3 aa, Artificial Sequence
  Synthetic polypeptide
GAS

SEQ ID NO: 51 - protein, 9 aa, Artificial Sequence
  Synthetic polypeptide
QQYRSWPIT

SEQ ID NO: 52 - protein, 8 aa, Artificial Sequence
  Synthetic polypeptide
GFTFTSYY

SEQ ID NO: 53 - protein, 8 aa, Artificial Sequence
  Synthetic polypeptide
ISGSGDST

SEQ ID NO: 54 - protein, 15 aa, Artificial Sequence
  Synthetic polypeptide
AREAGGDYDSGAFDI

SEQ ID NO: 55 - protein, 12 aa, Artificial Sequence
  Synthetic polypeptide
QSVLYSGNNKNY

SEQ ID NO: 56 - protein, 3 aa, Artificial Sequence
  Synthetic polypeptide
GAS

SEQ ID NO: 57 - protein, 9 aa, Artificial Sequence
  Synthetic polypeptide
QQYDYAPFT

SEQ ID NO: 58 - protein, 8 aa, Artificial Sequence
  Synthetic polypeptide
GFTFSSYA

SEQ ID NO: 59 - protein, 8 aa, Artificial Sequence
  Synthetic polypeptide
IDGEGGYT

SEQ ID NO: 60 - protein, 20 aa, Artificial Sequence
  Synthetic polypeptide
AREGVDYDILTGYYPYGMDV

SEQ ID NO: 61 - protein, 12 aa, Artificial Sequence
  Synthetic polypeptide
QSVLDSSNNKNY

SEQ ID NO: 62 - protein, 3 aa, Artificial Sequence
  Synthetic polypeptide
DAS

SEQ ID NO: 63 - protein, 9 aa, Artificial Sequence
  Synthetic polypeptide
QQGTSSPLT

SEQ ID NO: 64 - protein, 24 aa, Artificial Sequence
  Synthetic polypeptide
GGGGSGGGGSGGGGSGGGSGGGGS

SEQ ID NO: 65 - protein, 29 aa, Artificial Sequence
  Synthetic polypeptide
GGGGSGGGGSGGGGSGGGGSGGGSGGGGS

SEQ ID NO: 66 - protein, 5 aa, Artificial Sequence
  Synthetic polypeptide
GGGGS

SEQ ID NO: 67 - protein, 10 aa, Artificial Sequence
  Synthetic polypeptide
GGGGSGGGGS

SEQ ID NO: 68 - protein, 254 aa, Artificial Sequence
  Synthetic polypeptide
EVQLLESGGGLVQPGGSLRLSCAASGFTFSDYQMSWVRQAPGKGLEWVSGIQGGGGSTYYADSVKGRFTISRDNSKNTLYLQMNSLRAEDTAVYYCAREMWRGDYYSGMDVWGQGTTVTVSSGGGGSGGGGSGGGSGGGGSDIVMTQSPDSLAVSLGERATINCKSSQSVLDSYNNENNLAWYQQKPGQPPKLLIYWASTRESGVPDRFSGSGSGTDFTLTISSLQAEDVAVYYCQQYTSEPITFGQGTKVEIK

SEQ ID NO: 69 - protein, 257 aa, Artificial Sequence
  Synthetic polypeptide
EVQLLESGGGLVQPGGSLRLSCAASGFTFTSYAMSWVRQAPGKGLEWVSGIDGSGGGTNYADSVKGRFTISRDNSKNTLYLQMNSLRAEDTAVYYCARAYYDILTGYPVDGMDVWGQGTTVTVSSGGGGSGGGGSGGGSGGGGSDIVMTQSPDSLAVSLGERATINCKSSQSVLSSYNNENNLAWYQQKPGQPPKLLIYAASTRESGVPDRFSGSGSGTDFTLTISSLQAEDVAVYYCQQYYSEPYTFGQGTKVEIK

SEQ ID NO: 70 - protein, 249 aa, Artificial Sequence
  Synthetic polypeptide
EVQLLESGGGLVQPGGSLRLSCAASGFTFTDYAMSWVRQAPGKGLEWVSDIDGSGGSTDYADSVKGRFTISRDNSKNTLYLQMNSLRAEDTAVYYCALELGATTVYWGQGTLVTVSSGGGGSGGGGSGGGSGGGGSDIVMTQSPDSLAVSLGERATINCKSSQSVLRSSNNKNNLAWYQQKPGQPPKLLIYAASTRESGVPDRFSGSGSGTDFTLTISSLQAEDVAVYYCQQYYREPLTFGQGTKVEIK

SEQ ID NO: 71 - protein, 247 aa, Artificial Sequence
  Synthetic polypeptide
EVQLLESGGGLVQPGGSLRLSCAASGFTFTSTQMSWVRQAPGKGLEWVSEISGYGGSTYYADSVKGRFTISRDNSKNTLYLQMNSLRAEDTAVYYCAKDTEVSGDAFDIWGQGTMVTVSSGGGGSGGGGSGGGSGGGGSEIVLTQSPGTLSLSPGERATLSCRASQSVDSSNLAWYQQKPGQAPRLLIYGASSRATGIPDRFSGSGSGTDFTLTISRLEPEDFAVYYCQQYRSWPITFGQGTKVEIK

SEQ ID NO: 72 - protein, 254 aa, Artificial Sequence
  Synthetic polypeptide
EVQLLESGGGLVQPGGSLRLSCAASGFTFTSYYMSWVRQAPGKGLEWVSGISGSGDSTSYADSVKGRFTISRDNSKNTLYLQMNSLRAEDTAVYYCAREAGGDYDSGAFDIWGQGTMVTVSSGGGGSGGGGSGGGSGGGGSDIVMTQSPDSLAVSLGERATINCKSSQSVLYSGNNKNYLAWYQQKPGQPPKLLIYGASTRESGVPDRFSGSGSGTDFTLTISSLQAEDVAVYYCQQYDYAPFTFGQGTKVEIK

SEQ ID NO: 73 - protein, 259 aa, Artificial Sequence
  Synthetic polypeptide
EVQLLESGGGLVQPGGSLRLSCAASGFTFSSYAMSWVRQAPGKGLEWVSEIDGEGGYTNYADSVKGRFTISRDNSKNTLYLQMNSLRAEDTAVYYCAREGVDYDILTGYYPYGMDVWGQGTTVTVSSGGGGSGGGGSGGGSGGGGSDIVMTQSPDSLAVSLGERATINCKSSQSVLDSSNNKNYLAWYQQKPGQPPKLLIYDASTRESGVPDRFSGSGSGTDFTLTISSLQAEDVAVYYCQQGTSSPLTFGQGTKVEIK